(19) United States
(12) Patent Application Publication
(10) Pub. No.: US 2023/0274792 A1
(43) Pub. Date: Aug. 31, 2023

(54) SYSTEM AND METHOD FOR PRIME EDITING EFFICIENCY PREDICTION USING DEEP LEARNING

(71) Applicant: INDUSTRY-ACADEMIC COOPERATION FOUNDATION, YONSEI UNIVERSITY, Seoul (KR)

(72) Inventors: Hyongbum Henry KIM, Seoul (KR); Hui Kwon KIM, Seoul (KR); Goo Sang YU, Incheon (KR)

(73) Assignee: INDUSTRY-ACADEMIC COOPERATION FOUNDATION, YONSEI UNIVERSITY, Seoul (KR)

(21) Appl. No.: 18/007,241

(22) PCT Filed: Jul. 28, 2021
(86) PCT No.: PCT/KR2021/009794

§ 371 Date: Jan. 27, 2023

(30) Foreign Application Priority Data
Jul. 29, 2020 (KR) 10-2020-0094684

(51) Int. Cl. G16B 25/00; G16B 40/20; C12N 15/10; G06N 3/0464

(52) U.S. Cl. CPC G16B 25/00; G16B 40/20; C12N 15/1082; C12N 15/1089; G06N 3/0464

(57) ABSTRACT

A system for predicting prime editing efficiency by using deep learning, including: an information input unit that receives an input of data on prime editing efficiency of a prime editor, a predictive model generator for generating prime editing efficiency predictive models by performing deep learning to learn a relationship between features affecting prime editing efficiency and prime editing efficiency, by using the data received from the information input unit, a candidate sequence input unit that receives an input of a candidate target sequence for prime editing; and an efficiency predictor for predicting prime editing efficiency by applying the candidate target sequence input into the candidate sequence input unit to an efficiency predictive model generated in the predictive model generator.

### TECHNICAL FIELD

Provided are a system for predicting prime editing efficiency by using deep learning, a method of building the system, a method of predicting prime editing efficiency by using the system, and a computer-readable recording medium in which a program for executing the method of predicting prime editing efficiency on a computer is recorded.

### BACKGROUND ART

Prime editing is a revolutionary new genome editing method capable of introducing genetic changes of virtually any size without donor DNA or double-strand breaks (DSBs) (Anzalone, A. V. et al. Search-and-replace genome editing without double-strand breaks or donor DNA. Nature 576, 149-157 (2019)). These changes not only include insertions, deletions, and all 12 possible point mutations but also combinations thereof.

A prime editor (PE) is basically composed of: a Cas9-nickase-reverse-transcriptase (RT) fusion protein and a prime editing guide RNA (pegRNA), wherein the pegRNA includes a guide sequence that recognizes a target sequence; a trans-activating CRISPR RNA (tracrRNA) that serves as a scaffold sequence; a primer binding site (PBS) needed to initiate reverse transcription; and a reverse transcription (RT) template that includes a desired genetic change and is homologous to the target sequence. Four types of prime editors have been developed: prime editor 1 (PE1), prime editor 2 (PE2), prime editor 3 (PE3), and prime editor 3b (PE3b).

In prime editing, editing efficiency may vary greatly depending on various conditions. Some studies are being conducted on factors affecting prime editing efficiency, but the studies are still in the early stages.

Therefore, the development of computational models that identify factors affecting prime editing efficiency and predict prime editing activity at a given target sequence will greatly facilitate prime editing.

### DESCRIPTION OF EMBODIMENTS

### Technical Problem

Provided is a system for predicting prime editing efficiency by using deep learning.

Provided is a method of building a system for predicting prime editing efficiency by using deep learning.

Provided is a method of predicting prime editing efficiency by using the system for predicting prime editing efficiency.

Provided is a computer-readable recording medium in which a program for executing the method of predicting prime editing efficiency on a computer is recorded.

### Solution to Problem

An aspect provides a system for predicting prime editing efficiency by using deep learning.

The system for predicting prime editing efficiency by using deep learning includes: an information input unit that receives an input of data on prime editing efficiency of a prime editor; a predictive model generator for generating prime editing efficiency predictive models by performing deep learning to learn a relationship between features affecting prime editing efficiency and prime editing efficiency, by using the data received from the information input unit; a candidate sequence input unit that receives an input of a candidate target sequence for prime editing; and an efficiency predictor for predicting prime editing efficiency by applying the candidate target sequence input into the candidate sequence input unit to the efficiency predictive model generated in the predictive model generator.

The present inventors constructed prime editing efficiency data sets by using 54,836 pairs of pegRNA-encoding sequences and corresponding target sequences through high-throughput experiments, extracted features related to prime editing efficiency by using the same, and constructed a system for predicting prime editing efficiency for a given target sequence.

The system for predicting prime editing efficiency includes an information input unit for receiving data on prime editing efficiency of a prime editor.

“Prime editing” is a genome editing method using 4th generation genetic scissors, which are capable of introducing genetic changes by cleaving one strand of DNA without a double strand break.

Prime editing is performed by a “prime editor (PE)”. Types of the prime editor include prime editor 1 (PE1), prime editor 2 (PE2), prime editor 3 (PE3), and prime editor 3b (PE3b), but are not limited thereto. In an embodiment, the prime editor may be prime editor 2 (PE2). The prime editor includes a Cas9 nickase-reverse transcriptase (RT) fusion protein and prime editing guide RNA (pegRNA). The term “prime editor”, used herein, may merely mean to include a Cas9 nickase-RT fusion protein, or it may mean to include both the Cas9 nickase-RT fusion protein and pegRNA. For example, when pegRNA is separately introduced into a cell, introducing a prime editor may mean introducing a Cas9 nickase-RT fusion protein only. That is, when pegRNA is already introduced into a cell, introduction of a prime editor means introduction of a Cas9 nickase-RT fusion protein only. In an embodiment, the prime editor may mean a Cas9 nickase-RT fusion protein. The Cas9 nickase may be Cas9 H850A.

A “Cas9 nickase” used in prime editing may be modified to nick a single strand of DNA.

“Prime editing efficiency” means genome editing efficiency by a prime editor. Prime editing efficiency may be calculated by a rate of occurrence of the intended edit by a prime editor and pegRNA at a target sequence without generation of an unintended mutant when prime editing is performed. The prime editing efficiency may be expressed as percentage.

“Data on prime editing efficiency” may be existing known data or data directly obtained by any method that may be appropriately adopted by those skilled in the art, and the method by which the data is obtained is not limited within the range that the data may generate a predictive model capable of predicting prime editing efficiency. In an embodiment, the data may be prime editing efficiency data analyzed by using pegRNA and its corresponding target sequence through a high-throughput experiment.

Specifically, the data on prime editing efficiency may be obtained by a method including: introducing a prime editor into a cell library including an oligonucleotide including a nucleotide sequence encoding pegRNA and a target nucleotide sequence targeted by the pegRNA; performing deep sequencing by using DNA obtained from the cell library into which the prime editor has been introduced; and analyzing prime editing efficiency from the data obtained by deep sequencing.

A “reverse transcriptase (RT)” is an enzyme that synthesizes a new complementary DNA from an RNA template.

“Prime editing guide RNA (pegRNA)” includes: a guide sequence that recognizes a target sequence; a trans-activating CRISPR RNA (tracrRNA) that serves as a scaffold sequence; a primer binding site (PBS) needed to initiate a reverse transcription; and a reverse transcription (RT) template that includes a desired genetic modification.

In the pegRNA, the guide sequence includes a sequence complementary to the target sequence in whole or in part.

The term “target sequence” means a nucleotide sequence targeted by pegRNA. The target sequence may be a sequence that is expected to be targeted by pegRNA. The target sequence may be some of the genomic sequences known in the art, or may be an arbitrarily designed sequence to be analyzed by those skilled in the art by using the system of the present disclosure.

“Oligonucleotide” means a substance in which several to hundreds of nucleotides are linked by phosphodiester bonds. A length of the oligonucleotide may be 100 nts to 300 nts, 100 nts to 250 nts, or 100 nts to 200 nts, but is not limited thereto, and may be appropriately adjusted by those skilled in the art.

The nucleotide sequence encoding pegRNA included in the oligonucleotide may include a guide sequence, an RT template sequence, a PBS sequence, and the like.

The target nucleotide sequence included in the oligonucleotide may include a protospacer adjacent motif (PAM) and an RT template binding region. The RT template binding region may include a sequence completely or partially complementary to an RT template.

The oligonucleotide may further include a barcode sequence. Accordingly, the oligonucleotide may include a sequence encoding pegRNA, a barcode sequence, and a target sequence targeted by the pegRNA. A number of the barcode sequence may be one, two, or more. The barcode sequence may be appropriately designed by those skilled in the art according to the purpose. For example, the barcode sequence may be such that each pair of pegRNA and its corresponding target sequence may be identified after performing deep sequencing.

The oligonucleotide may further include an additional sequence to which primers may be bound for PCR amplification.

A “library” means a group (pool or population) including two or more substances of the same kind with different properties. Thus, an oligonucleotide library may be a group including two or more oligonucleotides differing in the nucleotide sequence, for example, two or more types of oligonucleotides differing in pegRNA and/or the target sequences. In addition, a cell library may be a group of two or more cells having different properties, for example, cells having different oligonucleotides included in the cells.

A “vector” may refer to a medium capable of delivering the oligonucleotide into a cell. Specifically, the vector may include an oligonucleotide including each sequence encoding pegRNA and a target sequence. The vector may be a viral vector, or a plasmid vector, but is not limited thereto. The viral vector may be a lentiviral vector, or a retroviral vector, but is not limited thereto. The vector may include essential regulatory elements operably linked to the insert so that the insert, that is, the oligonucleotide may be expressed, when the vector is introduced into a cell of a subject. The vector may be prepared and purified by using standard recombinant DNA techniques. A type of the vector is not particularly limited as long as the vector may function in desired cells such as prokaryotic cells and eukaryotic cells. The vector may include a promoter, an initiation codon, and a termination codon. In addition, DNA encoding a signal peptide, and/or an enhancer sequence, and/or 5′ or 3′ untranslated region in the desired gene, and/or a selectable marker region, and/or a replicable unit may also be appropriately included.

For a method of delivering the vector into a cell for preparation of a library, various methods known in the art may be used. Various methods known in the art may be performed, such as calcium phosphate-DNA co-precipitation method, diethylaminoethyl (DEAE)-dextran-mediated transfection method, polybrene-mediated transfection method, electroporation method, microinjection method, liposome fusion method, lipofectamine and protoplast fusion method, and the like. Furthermore, when a viral vector is used, infection of the virus particles may be used as a means to deliver the object, that is, a vector into the cells. In addition, the vector may be introduced into the cell by gene gun bombardment and the like. The introduced vector may exist in the cell as a vector itself or may be integrated into the chromosome, but its manner of existence is not limited thereto.

A type of cells into which the vector may be introduced may be appropriately selected by a person skilled in the art depending on the type of the vector and/or the type of desired cells, but examples may include bacterial cells such as Escherichia coli, Streptomyces, and Salmonella typhimurium; yeast cells; fungal cells such as Pichia pastoris; insect cells such as Drosophila and Spodoptera Sf9 cells; animal cells such as Chinese hamster ovary (CHO) cells, SP2/0 (mouse myeloma), human lymphoblastoid, COS, NSO (mouse myeloma), 293T, bowes melanoma cells, HT-1080, baby hamster kidney (BHK) cells, human embryonic kidney cells (HEK), and PERC.6 (human retinal cell); or plant cells.

The cell library prepared herein refers to a cell group into which an oligonucleotide including a pegRNA-encoding sequence and a target sequence, is introduced. In this regard, an oligonucleotide having a different pegRNA-encoding sequence and/or target sequence may be introduced into each cell.

A prime editor may be introduced into the cell library to induce prime editing. The prime editor may mean a Cas9 nickase-RT fusion protein. The prime editor may be introduced into a cell by a vector or may be introduced into a cell by itself, and a method of introduction is not limited as long as the prime editor may exhibit activity in the cell. In this regard, the description of the vector is as described above.

In the cell library, prime editing may occur by the introduced oligonucleotide including pegRNA and target sequence, and the introduced prime editor. That is, gene editing may occur for the introduced target sequence.

A method of obtaining DNA from a cell library into which the prime editor is introduced may be performed by using various DNA separation methods known in the related art.

Since gene editing is expected to have occurred at the introduced target sequence in each cell constituting the cell library, the target sequence may be sequenced to detect gene editing efficiency. The sequence analysis method is not limited to a specific method within the range that prime editing efficiency data may be obtained, but for example, deep sequencing may be used.

Analyzing prime editing efficiency from the data obtained by deep sequencing may include calculating prime editing efficiency.

Prime editing efficiencies may vary depending on types and/or lengths of a pegRNA sequence and a target sequence.

The data on prime editing efficiency may be provided as a data set.

The “information input unit” is a component that receives an input of the above-described prime editing efficiency data. The information input unit may directly receive an input of prime editing efficiency data from a user of the system or receive previously stored efficiency data, but is not limited thereto.

The system may further include a storage unit for storing previously obtained prime editing efficiency data or known prime editing efficiency data, but is not limited thereto. When the storage unit is included, the information input unit may receive an input of data of a set size or range from the storage unit and use the data to predict prime editing efficiency.

In an embodiment, the system may further include a database in which prime editing efficiency data is stored. The information input unit may receive an input of prime editing efficiency data from the database, but is not limited thereto.

The system for predicting prime editing efficiency includes a predictive model generator that generates prime editing efficiency predictive models by performing deep learning to learn a relationship between features affecting prime editing efficiency and prime editing efficiency by using the data received from the information input unit.

The “predictive model generator” refers to a component capable of learning the relationship between the features affecting prime editing efficiency and the prime editing efficiency by using the prime editing efficiency data input through the information input unit. The predictive model generator generates predictive models based on the learned information. Accordingly, a user may predict prime editing efficiency by using the predictive models.

The features affecting prime editing efficiency may be extracted from information about factors involved in prime editing. The factors involved in prime editing may include components constituting a prime editor and the target sequence. The components constituting the prime editor may include a Cas9-nickase, a reverse transcriptase, and pegRNA.

In an embodiment, the features affecting prime editing efficiency may be extracted from information about pegRNA and a target sequence.

The information about the pegRNA and the target sequence may include at least one of information about an RT template sequence, information about a PBS sequence, and information about the target sequence. Specifically, the information about the pegRNA and the target sequence may include at least one information selected from: a length of the RT template; specific sequence of the RT template; editing type; editing position; edited length; length of PBS; specific sequence of PBS; specific nucleotide sequence of the target sequence; melting temperature; GC count; minimum self-folding free energy of the target sequence, PBS and RT template sequence; and indel frequency related to Cas9-sgRNA activity in the target sequence, and information about any feature that may affect prime editing efficiency may be included without limitation in the type.

The editing type may include, but is not limited to, substitution, insertion, and deletion. The editing type may differ according to a type (for example, A, G, C, T) or number (for example, 1 nt, 2 nts, 3 nts) of nucleotides to be substituted, inserted, or deleted in the target sequence.

The editing position may be calculated based on a nicking site. For example, the editing position may be expressed as +1, +2, +3, etc. from a nicking site.

The “nicking site” refers to a site that is cleaved by a Cas9-nickase in a target sequence.

“Deep learning” is an artificial intelligence (AI) technology that allows computers to think and learn like humans, and is a technology that enables machines to learn and solve complex nonlinear problems on their own based on the artificial neural network theory. By using the deep-learning technology, a computer may recognize, infer, and judge by itself even when a person does not set all the judgment standards, and the technology may be widely used in voice and image recognition and photo analysis. In other words, deep learning may be defined as a set of machine learning algorithms that attempts a high level of abstraction (a task of summarizing key contents or functions in large amounts of data or complex data) through a combination of various nonlinear transformation methods.

The features affecting prime editing efficiency may be known features affecting prime editing efficiency, or may be features extracted by analyzing the prime editing efficiency data. The features affecting prime editing efficiency may be extracted by the predictive model generator, or features extracted by performing a separate method may be used. The separate method may be to perform an evaluation of feature importance by using the prime editing efficiency data, but is not limited thereto. For example, the evaluation of feature importance may use the Tree SHAP method, but is not limited thereto.

The predictive model generator may perform deep learning based on a convolutional neural network (CNN) or a multilayer perceptron (MLP).

In an embodiment, the features affecting prime editing efficiency may be a PBS length and a RT template length. Therefore, the predictive model generator may generate a prime editing efficiency predictive model by performing deep learning for learning the relationship between the PBS length and the RT template length and the prime editing efficiency based on the convolutional neural network by using the data input from the information input unit.

In an embodiment, the features affecting prime editing efficiency may further include melting temperature, GC count, GC content, minimum self-folding free energy, and the like.

The predictive model generator may convert data about nucleotide sequences among data input from the information input unit into a 4-dimensional binary matrix. The conversion to a 4-dimensional binary matrix may be performed by one-hot encoding.

The predictive model may include a convolutional layer and a fully connected layer.

The predictive model may include a convolutional layer, a fully connected layer, and a regression output layer.

Performing deep learning based on the convolutional neural network may include: obtaining two embedding vectors from the target sequence, the RT template and the PBS sequence through a convolution layer, and linking the embedding vectors with features affecting prime editing efficiency; multiplying a rectified-linear-unit (ReLU) activation function to the vector through a fully connected layer; and calculating a prediction score for prime editing efficiency by performing a linear transformation of an output through a regression output layer.

The predictive model may not include a pooling layer.

In an Example, deep learning was performed to learn the relationship between a PBS length and RT template length and prime editing efficiency based on a convolutional neural network using the prime editing efficiency data obtained by using a cell library having 48,000 pairs of pegRNAs and target sequences. As a result, a model DeepPE capable of predicting prime editing efficiency for a given target sequence was generated. Using the DeepPE, when a specific type of editing is intended in a given target sequence, the prime editing efficiency could be predicted according to the PBS length and RT template length.

In another embodiment, the features affecting prime editing efficiency may be an editing type, an editing position, or a combination thereof. Therefore, the predictive model generator may generate a prime editing efficiency predictive model by performing deep learning for learning the relationship between the editing type, editing position, or combination thereof and the prime editing efficiency based on a multilayer perceptron by using the data input from the information input unit.

In an Example, deep learning was performed to learn the relationship between the editing type or editing position, and the prime editing efficiency based on a multilayer perceptron by using the prime editing efficiency data obtained by using a cell library having 6,800 pairs of pegRNAs and target sequences. As a result, models PE_type and PE_position capable of predicting prime editing efficiency for a given target sequence were generated. By using the PE_type and PE_position, it was possible to predict prime editing efficiency according to editing types and/or editing positions in a given target sequence.

By using the same principle, when a specific editing type is intended in an arbitrary target sequence, a model capable of predicting prime editing efficiency according to a specific value of each feature affecting prime editing efficiency may be created.

The predictive model generator may include a feature extraction module for extracting features affecting prime editing efficiency from information about pegRNA and a target sequence, but is not limited thereto. In addition, the predictive model generator may further include a combination module combining features extracted from the feature extraction module, but is not limited thereto.

The system for predicting prime editing efficiency includes a candidate sequence input unit that receives an input of a candidate target sequence for prime editing.

The “candidate sequence input unit” is a component of the system for predicting prime editing efficiency for receiving an input of a candidate target sequence.

The candidate target sequence refers to a target nucleotide sequence of pegRNA whose prime editing efficiency is to be analyzed or predicted. The candidate target sequence may be derived from the genome sequence of a subject whose prime editing efficiency is to be confirmed, or may be any sequence designed and synthesized by a method known in the art, but its type is not limited within the range that the sequence may be applied to the system of the present disclosure to predict prime editing efficiency.

In an embodiment, the candidate target sequence may consist of 10 to 100, 20 to 100, 30 to 100, 10 to 90, 20 to 90, 30 to 90, 10 to 80, 20 to 80, 30 to 80, 10 to 70, 20 to 70, 30 to 70, 10 to 60, 20 to 60, 30 to 60, 10 to 50, 20 to 50, or 30 to 50 nucleotides, but is not limited thereto.

The candidate target sequence may include, but is not limited to, a protospacer adjacent motif (PAM), and a protospacer sequence. The PAM and protospacer sequences are sequences involved in a process in which a target sequence is recognized by a prime editor.

The system for predicting prime editing efficiency includes an efficiency predictor for predicting prime editing efficiency by applying a candidate target sequence input into the candidate sequence input unit to the efficiency predictive model generated in the predictive model generator.

The “efficiency predictor” is a component that predicts prime editing efficiency by applying the candidate target sequence input through the candidate sequence input unit to an efficiency predictive model built by a preset method.

In the system, the efficiency predictor may predict prime editing efficiency of the candidate target sequence by a prime editor.

In an Example, for a specific target sequence entered into DeepPE, when a specific type of editing is intended, prime editing efficiency according to a RT template length and a PBS length was predicted.

In another Example, prime editing efficiency was predicted according to an editing type (for example, editing type, editing position, number of edited nucleotides, etc.) for a specific target sequence input in PE_type and PE_position.

Therefore, a user of the system may design pegRNA sequences, specifically, RT templates and/or PBS sequences, for inducing gene editing in a given target sequence with reference to the prime editing efficiency predicted by the predictive model.

The system for predicting prime editing efficiency may further include an output unit for outputting the prime editing efficiency predicted by the efficiency predictor.

The information on prime editing efficiency output by the output unit may be represented by a calculated value of the prime editing efficiency or a relative value to a preset reference value, but a form or type of the output information is not limited. For example, the information on prime editing efficiency may be output visually or audibly.

Another aspect provides a method of building a system for predicting prime editing efficiency by using deep learning.

The method of building a system for predicting prime editing efficiency by using deep learning includes: obtaining a prime editing efficiency data set of a prime editor; and generating a prime editing efficiency predictive model by performing deep learning to learn a relationship between features affecting prime editing efficiency and prime editing efficiency, by using the prime editing efficiency data set.

The process of obtaining a prime editing efficiency data set may include: introducing a prime editor into a cell library including a nucleotide sequence encoding pegRNA and a target nucleotide sequence targeted by the pegRNA; performing deep sequencing by using DNA obtained from the cell library into which the prime editor has been introduced; and analyzing prime editing efficiency from the data obtained by deep sequencing.

The oligonucleotide may further include a barcode sequence. A description of the barcode sequence is as described above.

The prime editing efficiency may be calculated by a rate of occurrence of intended edits by a prime editor and pegRNA at a target sequence without generation of an unintended mutant.

The features affecting prime editing efficiency may be extracted from information about the pegRNA and the target sequence. Descriptions of “features affecting prime editing efficiency” and “information on pegRNA and target sequence” are as described above.

The information about the pegRNA and the target sequence may include at least one of information about an RT template sequence, information about a PBS sequence, and information about a target sequence, but is not limited thereto.

Deep learning may be performed based on a convolutional neural network (CNN) or a multilayer perceptron (MLP), in the process of generating a predictive model.

After the process of generating a predictive model, verifying the generated predictive model may be further included. The verification may be performed by a method known in the art.

Another aspect provides a method of predicting prime editing efficiency.

The method of predicting prime editing efficiency includes: designing candidate target sequences for prime editing; and predicting prime editing efficiency by applying the designed candidate target sequences to the system for predicting prime editing efficiency according to an aspect.

Descriptions of the candidate target sequence and the system for predicting prime editing efficiency are as described above.

Another aspect provides a computer readable recording medium on which is recorded a program for executing the method of predicting prime editing efficiency by a computer.

The program may implement the system for predicting prime editing efficiency or the method of predicting prime editing efficiency in a computer programming language.

The computer programming language capable of implementing the program may be Python, C, C++, Java, Fortran, Visual Basic, and the like, but is not limited thereto. The program may be stored in a recording medium such as a USB memory, a compact disc read only memory (CDROM), a hard disk, a magnetic diskette, or a similar medium or device, and may be connected to an internal or external network system. For example, a computer system may access a sequence database such as GenBank (http://www.ncbi.nlm.nih.gov/nucleotide) by using HTTP, HTTPS, or XML protocols, and search a nucleic acid sequence of a target gene and a regulatory region of the gene.

The program may be provided online or offline.

### Advantageous Effects of Disclosure

The system for predicting prime editing efficiency by using deep learning according to an aspect may predict prime editing efficiency with higher accuracy than existing machine learning-based prediction methods. Therefore, the system may be useful in all fields where genetic scissors are applied, such as disease treatment by gene editing.

### BRIEF DESCRIPTION OF DRAWINGS

FIG. 1 is a schematic diagram showing components of prime editing. A PE2 protein was expressed by transient transfection. A human U6 promoter (hU6) was used for expression of a pegRNA that guides PE2 to the target sequence. Guide, guide sequence; RTT, RT template; PBS, primer binding site; RT, reverse transcriptase; BSD-R, blasticidin resistance gene.

FIG. 2 shows configurations of Library 1 and Library 2. In Library 1, for 2,000 guide sequences, 24 combinations of different PBS lengths and RT template lengths were generated to construct 48,000 pegRNAs. In Library 2, 2,000 guide sequences were ligated with 34 different combinations of PBS and RT templates to generate various types of editing at different positions, resulting in 6,800 pegRNAs.

FIG. 3 is a schematic diagram showing how positions are specified within pegRNA, cDNA and a wide target sequence. Positions in pegRNA and cDNA generated from pegRNA are numbered starting from a nicking site of a Cas9 nickase. Positions in the wide target sequence were designated such that the 20th nucleotide upstream from PAM is position 1 and nucleotides of NGG PAM are at positions 21 to 23.

FIG. 4 is a schematic diagram of a high-throughput evaluation procedure of prime editing efficiency.

FIG. 5 shows a correlation of PE efficiencies of replicates independently transfected with PE2-encoding plasmids in two different experiments. Results from Library 1 and Library 2 are combined. To increase accuracy of the analysis, pairs of pegRNAs and target sequences were removed when the number of deep sequencing reads was less than 200 or the background prime editing frequency was 5% or more.

FIG. 6 shows a correlation between PE efficiency measured at endogenous sites and PE efficiency at corresponding integrated target sequences. A PE3 efficiency data set published in an earlier study (Anzalone, A. V. et al. Search-and-replace genome editing without double-strand breaks or donor DNA. Nature 576, 149-157 (2019)) is used.

FIG. 7 shows a correlation between PE efficiency measured at endogenous sites and PE efficiency at corresponding integrated target sequences. The data sets used were Endo-BR1-TR1, Endo-BR1-TR2, Endo-BR2-TR1, Endo-BR2-TR2, Endo-BR2-TR3, or Endo-BR3.

FIG. 8 shows a correlation between SpCas9-induced indel frequency and PE2 efficiency determined at the same target sequence. To minimize the effects of PBS lengths and RT template lengths, among 24 pegRNAs with different PBS lengths and RT template lengths, the pegRNA with the highest efficiency was selected for each target sequence. The number of pairs of pegRNAs and target sequences was n=1,956.

FIG. 9 shows a correlation between SpCas9-induced indel frequency and PE2 efficiency determined at the same target sequence by using Library 1. The correlation was evaluated considering all 24 combinations of PBS lengths and RT template lengths. The number of pairs of pegRNAs and target sequences was n=21,288.

FIG. 10 shows effects of PBS lengths and RT template lengths on PE2 efficiency. The heatmap represents average editing efficiency for a given PBS length and RT template length.

FIG. 11 shows effects of PBS lengths and RT template lengths on prime editing efficiency. (A) PE efficiency with PBSs of various lengths when the length of an RT template is fixed at 12 nt; (B) PE efficiency with RT templates of various lengths when the length of PBS is fixed at 13 nt. Subsets of the experimental group with no statistically significant difference (P<0.05) in the PE efficiency are indicated by the letters a, b, c, and d. In the boxes, the top, middle, and bottom lines represent the 25, 50, and 75 percentiles, respectively, whiskers represent the 10 and 90 percentiles, and outliers are indicated by individual dots. The number of pairs of pegRNAs and target sequences per experimental group assigned on the X axis is n=1,772 to 1,826.

FIG. 12 shows frequencies of pegRNAs with PE2 efficiency of higher than 5% for a given PBS length and RT template length.

FIG. 13 shows (A) frequencies of pegRNAs with editing efficiency of less than 5% for a given PBS length and RT template length; and (B) frequencies of pegRNAs with editing efficiency of higher than 5% for a given PBS length and RT template length.

FIG. 14 shows frequencies of combinations of PBS lengths and RT template lengths that induce the highest editing efficiency for a given target sequence.

FIG. 15 shows average editing efficiencies when combinations of PBS lengths and RT template lengths that showed the highest editing efficiency for each target are selected.

FIG. 16 shows the 10 most important features associated with PE2 efficiency determined by Tree SHAP (XGBoost classifier). In the graph on the right, each target sequence is indicated by a dot; and the position of the point on the X-axis represents a SHAP value. High and low SHAP values are associated with high and low prime editing efficiency, respectively. The color of the dots indicates a value of the relevant feature for a particular target sequence; and red and blue respectively indicate high and low values of the relevant feature. The overlapped points are slightly separated in the Y-axis direction to clarify the density.

FIG. 17 shows the 1st to 51st most important features associated with PE2 efficiency, as determined by the Tree SHAP.

FIG. 18 shows the 52nd to 100th most important features associated with PE2 efficiency, as determined by the Tree SHAP.

FIG. 19 shows effects of GC content and GC count in PBS and an RT template on prime editing efficiency.

FIG. 20 shows effects of a melting temperature of PBS, and a target DNA region that corresponds to an RT template, on prime editing efficiency. The PBS and RT template were 13 nt and 12 nt in length, respectively. The number of pairs of pegRNAs and target sequences per experimental group assigned on the X-axis was n=13 to 736.

FIG. 21 shows PE2 efficiencies for insertions, deletions, and substitutions of 1-bp. The numbers of pairs of pegRNAs and target sequences were 739 for insertions, 178 for deletions, and 566 for substitutions.

FIG. 22 shows effects of types and numbers of inserted nucleotides on PE2 efficiency. The numbers of pairs of pegRNAs and target sequences were 183, 183, 188, 185, 184, 179, and 163 for insertions of A, C, G, T, AG, AGGAA (5 bp), and AGGGAATCATG (10 bp), respectively.

FIG. 23 shows effects of deletion lengths on PE2 efficiency. The numbers of pairs of pegRNAs and target sequences were 178, 189, 185, and 169 for deletions of 1 bp, 2 bp, 5 bp, and 10 bp, respectively.

FIG. 24 shows effects of substitution types on PE2 efficiency. The numbers of pairs of pegRNAs and target sequences were 88, 87, 36, 35, 34, 44, 21, 20, 45, 45, 90, and 21, for C to T conversion, C to G conversion, A to G conversion, A to C conversion, A to T conversion, G to T conversion, and T to A conversion, respectively.

FIG. 25 shows effects of substitution types on prime editing efficiency. The numbers of pairs of pegRNAs and target sequences were respectively 52, 40, 50, and 35, for A to T conversion, C to G conversion, G to C conversion, and T to A conversion (left graph), 49, 44, 43, and 42, for A to T conversion, C to G conversion, G to C conversion, and T to A conversion (middle graph), and 29, 46, 51, and 47, for A to T conversion, C to G conversion, G to C conversion, and T to A conversion (right graph).

FIG. 26 shows effects of editing positions on PE2 efficiency for substitutions of 1-bp conversions. Editing positions shown on the X-axis are counted from the nicking site. The numbers of pairs of pegRNAs and target sequences were respectively 179, 186, 184, 180, 173, 184, 182, 178, 177, 178, and 173 for positions +1, +2, +3, +4, +5, +6, +7, +8, +9, +11, and +14.

FIG. 27 shows effects of editing positions on prime editing efficiency for substitutions of 1-bp conversions at two positions. The numbers of pairs of pegRNAs and target sequences were 190, 181, 186, 190, 177, 180, 183, 170, and 169 for positions +1 and +2, positions +1 and +5, positions +1 and +10, positions +2 and +3, positions +2 and +5, positions +2 and +10, positions +5 and +6, positions +5 and +10, and positions +10 and +11, respectively.

FIG. 28 shows relative partial editing frequencies according to distances between the two editing positions described in FIG. 27.

FIG. 29 shows results of an analysis of prime editing when two nucleotides are to be substituted. The heatmap shows average frequencies of partial (1 nt) and total (2 nt) editing. The numbers of pairs of pegRNAs and target sequences were 190, 181, 186, 190, 177, 180, 183, 170, and 169, for positions +1 and +2, positions +1 and +5, positions +1 and +10, positions +2 and +3, positions +2 and +5, positions +2 and +10, positions +5 and +6, positions +5 and +10, and positions +10 and +11, respectively.

FIG. 30 shows cross-validation results of predictive models according to machine learning frameworks used.

FIG. 31 shows evaluation results of DeepPE using the data sets HT-Test (number of pairs of pegRNAs and target sequences n=4,457) and Endo-BR1-TR1 (n=26).

FIG. 32 is results of comparing the performance of DeepPE with that of other prediction models using the dataset HT-Test. The bar graph represents the Spearman correlation coefficient between the measured PE2 efficiency and the predicted activity score. The number of pairs of pegRNAs and target sequences was n=4,457.

FIG. 33 shows evaluation results of DeepPE using six data sets obtained by measuring PE2 efficiencies at endogenous sites after transient transfection of HEK293T cells with plasmids encoding pegRNA and PE2. The numbers of target sequences were 26, 25, 23, 23, 23, and 16, for data sets Endo-BR1-TR1, Endo-BR1-TR2, Endo-BR2-TR1, Endo-BR2-TR2, Endo-BR2-TR3, and Endo-BR3, respectively.

FIG. 34 shows evaluation results of DeepPE using HCT116 and MDA-MB-231 cells. Eight data sets of PE2 efficiency were generated by using HCT116 (abbreviated as HCT) and MDA-MB-231 (abbreviated as MDA) cell lines with lentiviral integrated target sequences that have never been used for training DeepPE. The numbers of pairs of pegRNAs and target sequences were 72, 75, 75, 75, 71, 73, 74, and 75, for HCT-BR1-TR1, HCT-BR1-TR2, HCT-BR2-TR1, HCT-BR2-TR2, MDA-BR1-TR1, MDA-BR1-TR2, MDA-BR2-TR1, and MDA-BR2-TR2, respectively. Two biological replicates (BR1 and BR2) were evaluated per cell line, and each biological replicate had two technical replicates (TR1 and TR2).

FIG. 35 shows performance comparisons of DeepPE and other methods for selecting the most efficient combination out of 24 possible combinations of PBS lengths and RT template lengths at a given target sequence. For example, “13-nt PBS & 12 nt-PT template” means selecting a combination of these lengths regardless of the target sequence. Recommendations A and B of an earlier study are based on using 13-nt PBS and 12-nt RT template (RTT) and not using G as the last template nucleotide by changing the RTT length as needed. In recommendation A, when the last template nucleotide is G, 10-nt RTT is chosen over 12-nt RTT. After such a change, when the last template nucleotide is G again, 15-nt RTT is selected. In recommendation B, when the last template nucleotide is G, 15-nt RTT is chosen over 12-nt RTT. After such a change, when the last template nucleotide is G again, 10-nt RTT is selected. As a control, pegRNAs were randomly selected (Random 1 and Random 2). The number of target sequences was 97 per group.

FIG. 36 shows cross-validation results of PE-type according to machine learning frameworks used.

FIG. 37 shows cross-validation results of PE_position according to machine learning frameworks used.

### BEST MODE

Hereinafter, the present disclosure will be described in more detail through examples. However, these examples are intended to illustrate the present disclosure, and the scope of the present disclosure is not limited to these examples.

### Example 1: Preparation of Materials

### Example 1-1: Construction of pLenti-PE2-BSD Vector Expressing Prime Editor 2 (PE2)

The vector expressing the genetic scissors prime editor 2 (PE2) was constructed as follows. The LentiCas9-Blast plasmid (Addgene #52962) was digested with Agel and BamHI restriction enzymes (NEB) at 37° C. for 4 hours and treated with 1 μl of Quick-CIP (NEB) at 37° C. for 10 minutes. Next, the linearized plasmid was gel-purified by using a MEGAquick-spin whole fragment DNA purification kit (iNtRON Biotechnology). The PE2-encoding sequence from pCMV-PE2 (Addgene #132775) was amplified by PCR by using a Solg™ 2× pfu PCR smart mix (Solgent). The amplicons were assembled into the linearized LentiCas9-Blast plasmids by using a NEBuilder HiFi DNA assembly kit (NEB). The assembled plasmids were named pLenti-PE2-BSD.

### Example 1-2: Design of Oligonucleotide Libraries

An oligonucleotide pool including 54,836 pairs of pegRNAs and target sequences was synthesized by Twist Bioscience (San Francisco, Calif.).

Each oligonucleotide contained the following components: a 19-nt guide sequence, BsmBI restriction site #1, a 15-nt barcode sequence (barcode 1), BsmBI restriction site #2, an RT template sequence, a primer binding site (PBS) sequence, a poly-T sequence, an 18-nt barcode sequence (barcode 2), and a corresponding wide target sequence of 43-nt to 47-nt including a protospacer adjacent motif (PAM) and an RT template binding region.

Barcode 1 is a stuffer that may be removed by cleaving with BsmBI. Barcode 2 (located upstream of the target sequence) allows individual pairs of pegRNAs and target sequences to be identified after deep sequencing. Oligonucleotides including unintended BsmBI restriction sites in their sequences were excluded.

In order to test effects of PBS lengths and RT template lengths on PE2 efficiency, pegRNAs having 24 combinations of PBS lengths and RT template lengths (6 PBS lengths (7, 9, 11, 13, 15, and 17 nucleotides (nts))×4 RT template lengths (10, 12, 15, and 20 nts)=24) were prepared, for 2,000 pairs of guide sequences and target sequences, resulting in a total of 48,000 (=24×2,000) pairs of pegRNAs and target sequences (Library 1). The pegRNA was designed to generate a G to C conversion mutation at position +5 from the nicking site. The 2,000 target sequences were randomly selected from human protein-encoding genes. Here, the indel frequencies induced by SpCas9 have been measured in a previous study (Kim, H. K. et al. SpCas9 activity prediction by DeepSpCas9, a deep learning-based model with high generalization performance. Sci Adv 5, eaax9249 (2019)), which makes it possible to determine the correlation between SpCas9 and PE efficiency in the same target sequence.

In addition, another library, named Library 2, was prepared to evaluate effects of gene editing positions, types, and lengths on PE2 efficiency. Specifically, 200 target sequences were randomly selected from the 2,000 target sequences used in Library 1, and 34 different RT templates for each target sequence were designed as follows.
i) Effects of editing positions (11 RT templates): RT templates were designed to introduce conversion mutations at positions +1, +2, . . . , +8, +9, +11, and +14 from the nicking site. The PBS length and RT template length were fixed at 13 nts and 20 nts, respectively.
ii) Effects of editing types and lengths (14 RT templates): RT templates were designed to introduce insertions (inserted sequences=A, G, C, T, AG, AGGAA, and AGGAATCATG), deletions (1-, 2-, 5-, and 10-nt), and single base substitutions (all possible 1-nt substitutions) at position +1 from the nicking site. The lengths of the right homology arms of the PBSs and RT templates were fixed at 13 nts and 14 nts, respectively.
iii) Effects of PAM editing (9 RT templates): RT templates were designed to introduce 2-bp conversion mutations at positions +1 and +2, +1 and +5, +1 and +10, +2 and +3, +2 and +5, +2 and +10, +5 and +6, +5 and +10, and +10 and +11. The PBS length and RT template length were fixed at 13 nts and 16 nts, respectively.

In addition, 36 pairs of pegRNAs and target sequences having 5 unique barcodes per target sequence used in an earlier prime editing study (Anzalone, A. V. et al. Search-and-replace genome editing without double-strand breaks or donor DNA. Nature 576, 149-157 (2019)) were included. This set was used to determine the correlation between integrated sequences and prime editing efficiency at endogenous sites.

Thus, a total of 54,836 pairs of pegRNAs and target sequences, obtained by adding 48,000 pairs (from Library 1, 2,000×24)+6,800 pairs (from Library 2, 200×34)+36 pairs (from an earlier prime editing study), were used.

### Example 1-3: Preparation of Plasmid Library

A plasmid library containing the pairs of pegRNA-encoding sequences and corresponding target sequences were prepared by using a two-step cloning process:

(Step I) Gibson assembly, and

(Step II) restriction enzyme-induced cleavage and ligation.

During oligonucleotide amplification via PCR, separation of paired guide RNAs and target sequences was effectively prevented by this two-step process. The multi-step procedure was adapted and modified from a previously reported method (Shen, J. P. et al. Combinatorial CRISPR-Cas9 screens for de novo mapping of genetic interactions. Nat Methods 14, 573-576 (2017)).

(1) Step I: Construction of an Initial Plasmid Library Containing Pairs of pegRNA-Encoding Sequences and Target Sequences

The oligonucleotide pool was amplified by 15 cycles of PCR by using the Phusion Polymerase (NEB) and then the amplicons were gel purified. Lenti_gRNA-Puro vectors (Addgene #84752) were digested with BsmBI enzymes (NEB) at 55° C. for 6 hours. The linearized vectors were treated with 1 μl of Quick CIP at 37° C. for 10 minutes and gel purified. The amplified pool of oligonucleotides was assembled with the linearized Lenti_gRNA-Puro vectors by using the Gibson assembly. After column purification, the electrocompetent cells (Lucigen) were transformed with the assembled product by using a MicroPulser (Bio-Rad). Subsequently, SOC medium (2 ml) was added to the transformation mixture and incubated at 37° C. for 1 hour. The cells were then seeded onto Luria-Bertani (LB) agar plates containing 50 μg/ml of carbenicillin and incubated. Small fractions (0.1 μl, 0.01 μl, and 0.001 μl) of the culture were separately seeded to allow determination of the library coverage. Plasmids were extracted from the total harvested colonies. The calculated coverage of this initial plasmid library was 113 times the number of the oligonucleotides.

(2) Step II: Insertion of sgRNA Scaffold

The initial plasmid library prepared in Step I was digested with BsmBI for 8 hours, and then treated with 1 μl of Quick CIP at 37° C. for 10 minutes. The digested product was subjected to size-selection on a 0.6% agarose gel, followed by gel purification. The sgRNA scaffold sequence in a pRG2 plasmid (Addgene #104174) was amplified by 30 cycles of PCR using a Phusion polymerase and primer pairs having BsmBI restriction sites in each member of the pairs. The resulting amplicons were digested with BsmBI for at least 12 hours and gel purified on a 2% agarose gel. The purified insert (10 ng) was ligated to initial plasmid library vectors (200 ng) which were digested for 16 hours at 16° C. by using T4 ligases (Enzynomics). The ligated product was column purified and electroporated into Endura electrocompetent cells (Lucigen). The colonies were harvested and the final plasmid library was extracted. The calculated coverage of the final plasmid library was 785x.

### Example 1-4: Production of Lentiviruses

HEK293T cells (4.0×10⁶ or 8.0×10⁶) were seeded in a 100-mm or 150-mm cell culture dish containing Dulbecco's modified eagle medium (DMEM). After 15 hours, DMEM was replaced with fresh medium containing 25 μM of chloroquine diphosphate, and the cells were incubated for an additional 5 hours. The plasmid library and psPAX2 (Addgene #12260) were mixed with pMD2.G (Addgene #12259) in a molar ratio of 1.3:0.72:1.64, and co-transfected into HEK293T cells by using polyethyleneimine. 15 hours after the transfection, the cells were refreshed with maintenance medium. 48 hours after the transfection, the lentivirus-containing supernatant was collected, filtered through a Millex-HV 0.45-μm low protein binding membrane (Millipore), aliquoted, and stored at −80° C. Serial dilutions of the viral aliquots were transduced into HEK293T cells in the presence of polybrene (8 μg/ml), to determine the viral titer. Untransduced cells and cells treated with serially diluted viral aliquots were cultured in the presence of 2 μg/ml of puromycin (Invitrogen). Virus titer was estimated by counting the number of viable cells in the virus-treated population when almost all non-transduced cells were dead.

### Example 1-5: Generation of Cell Library

To prepare for lentiviral transduction, HEK293T cells were seeded in nine 150-mm dishes (density of 1.6×10⁷ cells per dish) and incubated overnight. The lentiviral library was transduced into cells at a multiplicity of infection (MOI) of 0.3 to achieve a coverage of 500 times or more compared to the initial number of oligonucleotides. Subsequently, the cells were incubated overnight and then maintained in 2 μg/ml of puromycin for 5 days to remove non-transduced cells. The cells in the cell library were maintained at a number of at least 3.0×10⁷ throughout the study, in order to preserve the diversity.

### Example 1-6: PE2 Delivery to Cell Library

A total of 3.0×10⁷ cells (three 150-mm culture dishes containing 1.0×10⁷ cells each) were transfected with pLenti-PE2-BSD plasmids (80 μg per dish) using 80 μl of Lipofectamine 2000 (Thermo Fisher Scientific) according to the manufacturers instructions. The culture medium was replaced with DMEM supplemented with 10% fetal bovine serum and 20 μg/ml blasticidin S (InvivoGen), 6 hours after the transfection. On day 4.8 after the transfection, the cells were harvested.

### Example 2: Experimental Method and Measurement of Results

### Example 2-1: Measurement of Efficiency of Prime Editor 2 (PE2) at Endogenous Sites

To validate the results of the high-throughput experiment, 33 individual pegRNA-encoding plasmids were randomly selected from the final plasmid library. To prepare for transfection, HEK293T cells were seeded in a 48-well plate at a density of 5.0×10⁴ cells per well or 1.0×10⁵ cells per well 16 to 18 hours prior to the transfection. The cells were transfected with a mixture of PE2-encoding plasmids (pLenti-PE2-BSD, 75 ng per 1.0×10⁴ cells) and pegRNA-encoding plasmids (25 ng per 1.0×10⁴ cells) according to the manufacturers instructions by using 1 μl of Lipofectamine 2000 or a TransIT-2020 transfection reagent per 1,000 ng of DNA. After overnight incubation, the culture medium was replaced with DMEM containing puromycin (2 μg/ml). The cells were harvested 4.5 days (for Endo-BR1 and Endo-BR2) or 7 days (Endo-BR3) after the transfection.

### Example 2-2: Measurement of PE2 Efficiency in HCT116 and MDA-MB-231 Cell Lines

HCT116 and MDA-MB-231 cells were sub-cultured in DMEM and RPMI supplemented with 10% (v/v) fetal bovine serum (FBS), respectively, at 37° C. in the presence of 5% CO₂. To generate PE2-expressing cell lines, HCT116 and MDA-MB-231 cells were transduced with PE2-encoding lentiviral vectors at a multiplicity of infection (MOI) of 0.3 in culture medium containing 8 μg/ml of polybrene. After overnight incubation, the cells were cultured in the presence of 10 μg/ml of blasticidin S for 7 days to remove non-transduced cells.

75 plasmids containing pairs of pegRNA-encoding sequences and corresponding target sequences were randomly selected from plasmid Library 1; and identity of the plasmids was determined by Sanger sequencing. Subsequently, a lentiviral library was generated from the plasmid pool. PE2-expressing HCT116 and MDA-MB-231 cells were seeded in 6-well plates at a density of 2.0×10⁵ cells per well, incubated overnight, and transduced with the lentiviral library. After overnight incubation, the culture medium was replaced with DMEM containing 1 μg/ml of puromycin and 10 μg/ml of blasticidin S, or RPMI containing 2 μg/ml of puromycin and 10 μg/ml of blasticidin S for HCT116 and MDA-MB-231 cell lines, respectively. 4.5 days after the transduction, the cells were harvested and analyzed.

### Example 2-3: Performing Deep Sequencing

Genomic DNA was extracted from the harvested cells by using a Wizard Genomic DNA purification kit (Promega).

For a high-throughput experiment, integrated barcodes and target sequences were PCR amplified by using 2X Taq PCR Smart mix (SolGent). For each cell library, the first PCR included a total of 400 μg of genomic DNA; and the coverage was expected to be 700-fold or greater than the library, assuming 10 μg of genomic DNA per 10⁶ cells. After performing 80 independent 50-μl PCR reactions with an initial genomic DNA concentration of 5 μg per reaction, the products were pooled and gel-purified with a MEGAquick-spin total fragment DNA purification kit (iNtRON Biotechnology). Subsequently, 100 ng of purified DNA was then amplified by PCR using primers including both the Illumina adapter and barcode sequence.

To measure PE2 efficiency at endogenous sites, an independent first PCR was performed in a 40-μL reaction volume including 200 ng of initial genomic DNA template per sample. A second PCR to attach the Illumina adapter and barcode sequence was then performed by using 20 ng of the purified product of the first PCR in a 30 μl reaction volume. After gel purification, the resulting amplicons were analyzed by using HiSeq or MiniSeq (Illumina, San Diego, Calif.).

### Example 2-4: Analysis of Prime Editing Efficiency

For analysis of deep sequencing data, Python scripts were used. Each pair of pegRNA and a target sequence was identified by a sequence of 22 nts (a barcode of 18 nts and a sequence of 4 nts located upstream of the barcode). Reads including specific edits without unintended mutations within the wide target sequence were considered to represent PE2-induced mutations. To exclude background prime editing frequencies arising from array synthesis and PCR amplification procedures, the background prime editing frequencies measured in the absence of PE2 were subtracted from the observed prime editing frequencies as shown below.

$$Prime editing efficiency ( \% ) = \frac{Number of reads with intended edits and specific barcode - ( total number of reads with specific barcode \times background prime editing frequency ) + 100}{Total number of reads with specific barcode - ( total number of reads with specific barcode \times background prime editing frequency ) + 100} \times 100$$

Deep sequencing data were filtered to improve accuracy of the analysis. Specifically, pairs of pegRNAs and target sequences with a deep sequencing read count of less than 200 and a background prime editing frequency of more than 5% were excluded.

### Example 2-5: Evaluation of Feature Importance

To measure feature importance for predicting PE2 efficiency, the Tree SHAP method (SHapley Additive explanations incorporated into XGBoost algorithm) was used (Lundberg, S. M. et al. From local explanations to global understanding with explainable Al for trees. Nature Machine Intelligence 2, 56-67 (2020)). Features and trained XGBoost models with the best hyperparameter configuration determined from 5-fold cross-validation were extracted. In the Tree SHAP method, each feature of the trained XGBoost model was assigned an importance score per sample. An importance score represents the feature's effect on the base value in the model output, and was calculated based on the game-theoretic Shapley value for optimal credit assignment. Distribution of SHAP values over the entire data set was shown or the mean absolute value was provided in order to provide an overall overview of feature importance in the model.

### Example 2-6: Development of Deep Learning-Based Computational Models

(1) Development of DeepPE

DeepPE is a deep learning-based computational model that predicts an optimal combination of PBS lengths and RT template lengths introducing a G to C conversion mutation at position +5 from the nicking site.

The present inventors used a training data set consisting of prime editing efficiencies induced by PE2 and 38,692 pegRNAs; these training data included a wide target sequence of 47 nts, RT templates+PBS sequence of 17 nts to 37 nts, and 20 additional features (for example, melting temperature, GC count, GC content, minimum self-folding free energy, etc.). Nucleotide sequences were converted into a 4-dimensional binary matrix by one-hot encoding.

DeepPE was developed by using convolutional layers and fully connected layers.

The convolutional layers obtained two embedding vectors from the wide target sequence and the RT template+PBS sequence by using 10 filters of 3-nt length. The embedding vectors were then linked with 20 biological features. Since a deep reinforcement learning algorithm was implemented to retain local information, a pooling layer was excluded.

A fully connected layer with 1,000 units multiplied the vectors with the rectified-linear-unit (ReLU) activation function.

The regression output layer performed a linear transformation of the output and calculated a prediction score for PE2 efficiency.

After testing nine different models (hyperparameters; number of filters (10, 20, and 40) and units (200, 500, and 1,000) for each convolutional layer and fully connected layer, respectively), and the model that showed the highest Spearman correlation coefficient between the experimentally measured activity level and the predicted activity level was selected during the 5-fold cross-validation. Dropout was used to avoid overfitting with a ratio of 0.3. The mean-squared error, as the objective function, and an Adam optimizer with a learning rate of 10-3 were used.

DeepPE was implemented by using TensorFlow.

(2) Development of PE_type and PE_position

PE_type is a deep learning-based computational model that predicts prime editing efficiency according to the editing type for a given target sequence.

PE_position is a deep learning-based computational model that predicts prime editing efficiency according to the editing position for a given target sequence.

To develop a deep learning-based algorithm for predicting PE2 efficiency for various edit types and positions, a multilayer perceptron (MLP) was used instead of a convolutional neural network. By performing cross-validation, selection was made from 18 MLP models with similar architecture and number of parameters to DeepPE but without a convolution. The considered hyperparameter configurations were: number of layers (chosen from [2, 3]), number of units in each hidden layer (chosen from [1000, 200, 50] for a first hidden layer, and chosen from [50] for a second hidden layer), dropout regularization parameter, learning rate (chosen from [0.01, 0.001, 0.0001]), and ReLU activation function.

### Example 2-7: Comparison with Existing Machine Learning-Based Models

(1) Generation of Data Subsets for Machine Learning

PE2 efficiency data obtained by using Library 1 were divided into HT-training and HT-test by stratified random sampling to ensure that the same target sequences were not shared between the two data sets. Similarly, PE2 efficiency data obtained by using Library 2 was divided into Type-training, Type-test, Position-training, and Position-test to ensure that the same target sequences were not shared between the training data sets and the test data sets. The target sequences used in the generation of the data sets Endo-BR1, Endo-BR2, Endo-BR3, HCT-BR1, HCT-BR2, MDA-BR1, and MDA-BR2 were included in the corresponding test data set, in order that the target sequences were not shared between the training data sets and the test data sets.

(2) Machine Learning-Based Model Training

Learning was conducted based on existing machine learning algorithms XGBoost, gradient-boosted regression tree, random forest, L1-regularized linear regression, L2-regularized linear regression, L1L2-regularized linear regression, and support vector machine (SVM) to compare the performance with that of DeepPE. The above models were implemented with a XGBoost Python package (ver 0.90) and scikit-learn (ver 0.19.1).

A total of 1,766 features were extracted from the wide target sequences and PBS and RT template sequences. The features included position-independent and position-dependent nucleotides and dinucleotides, melting temperatures, GC counts, minimum self-folding free energies of wide target sequence, PBS and RT template sequences, and DeepSpCas9 scores (Kim, H. K. et al. SpCas9 activity prediction by DeepSpCas9, a deep learning-based model with high generalization performance. Sci Adv 5, eaax9249 (2019)). The melting temperature was calculated by a program (https://biopython.org/docs/1.74/api/Bio.SeqUtils.MeltingTemp.html) using default settings without considering the cell nucleus environment. For model selection among regularization parameters and hyperparameter configurations, 5-fold cross-validation was performed.

For XGBoost and gradient boosted regression tree, over 144 models selected from the following hyperparameter configurations were searched: number of base estimators (chosen from [5, 10, 50, 100]), maximum depth of an individual regression estimator (chosen from [5, 10, 50, 100]), minimum number of samples at a leaf node (chosen from [1,2,4]), learning rate (chosen from [0.05, 0.1, 0.2]).

For random forest, over 144 models selected from the hyperparameter configurations as listed above except for the learning rate were searched; and the maximum number of features to consider when looking for the best split were searched (chosen from [all features, square root of all features, binary logarithm of all features]).

For L1-, L2-, and L1L2-regularized linear regression, over 144 points were searched that were evenly spaced between 10⁻⁶ and 10⁶ in the log space to optimize the regularization parameters.

For SVM, over 144 models were searched from the following hyperparameters: penalty parameter C and kernel parameter γ, 12 points that were evenly spaced between 10⁻³ and 10³.

### Example 2-8: Statistical Significance

To compare prime editing efficiencies between experiments using different pegRNAs, Tukey's post hoc test followed by one-way ANOVA was used. To compare Spearman correlations between prediction scores of predictive models, Steiger's test, which is a method of testing two dependent correlation coefficients in exactly the same data set, was used. A chi-square test was performed to determine the relationship between these two parameters when the most efficient combination of PBS length and RT template length per target sequence was selected. In order to improve accuracy of the chi-square analysis, target sequences exhibiting prime editing efficiency of less than 10% were filtered out from the analysis, even when the most efficient combination of the two parameters was selected. The two-tailed paired t-test was used to compare PE2 efficiencies of pegRNAs with PBS lengths and RT template lengths selected by using DeepPE or using recommendations of earlier studies at a given target sequence. PASW Statistics (version 18.0, IBM) and Microsoft Excel (version 16.0, Microsoft Corporation) were used to determine statistical significance.

### Example 2-9: Data Availability

The deep-sequencing data from this study are submitted to the National Center for Biotechnology Information (NCBI) Sequence Read Archive (SRA; https://www.ncbi.nlm.nih.gov/sra/) under accession no. SRR11529289.

### Experimental Example 1: Collection of Prime Editing Efficiency Data

The paired library approach was used for a high-throughput analysis of PE2 efficiency.

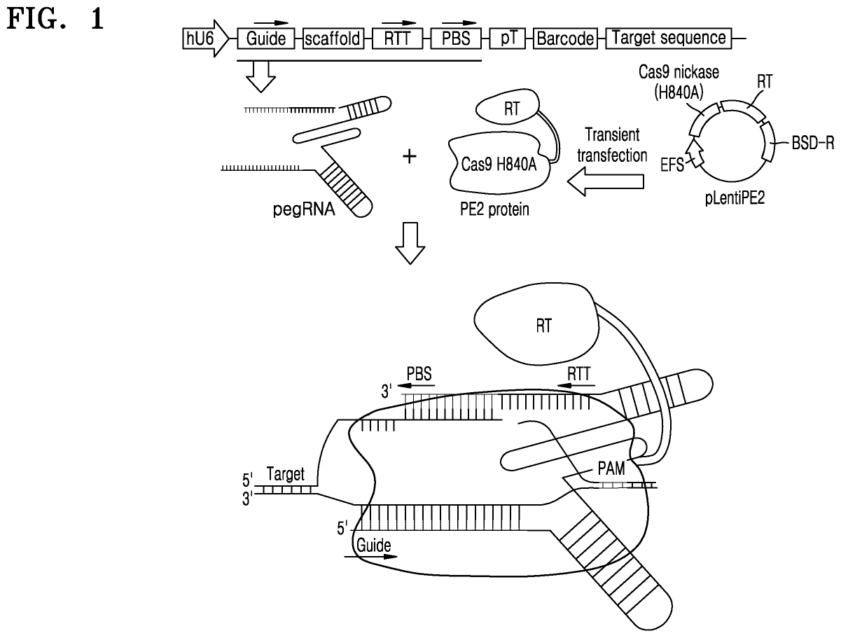

FIG. 1 is a schematic diagram showing components of prime editing.

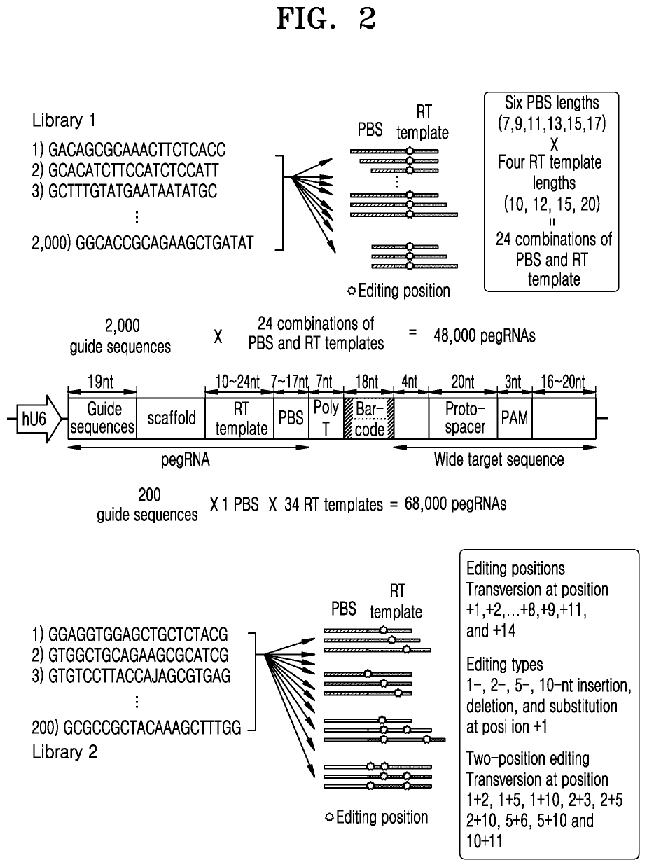

FIG. 2 shows configurations of Library 1 and Library 2.

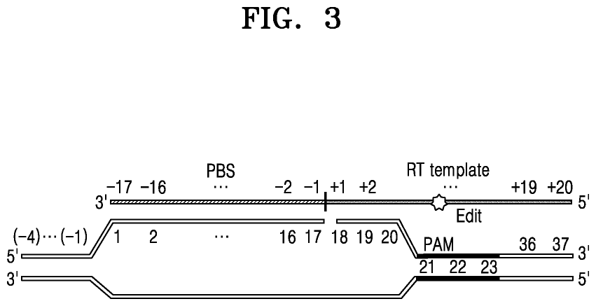

FIG. 3 is a schematic diagram showing how positions are specified within pegRNA, cDNA and wide target sequence.

The present inventors prepared a lentiviral plasmid library, termed Library 1, from an oligonucleotide pool including 48,000 pairs of pegRNA-encoding sequences and corresponding target sequences (=2,000 target sequences×24 combinations of PBS and RT template/target sequences).

To test the effect of PBS lengths and RT template lengths on PE2 efficiency, the library included 24 combinations of different PBS lengths and RT template lengths (6 PBS lengths (7 nts, 9 nts, 11 nts, 13 nts, 15 nts, and 17 nts)×4 RT template lengths (10 nts, 12 nts, 15 nts, and 20 nts)=24 combinations) for 2,000 pairs of guide and target sequences, that induce a G to C conversion mutation at position +5 from the nicking site (position 22 within the wide target sequence). That is, the library includes 48,000 (=24×2,000) pairs of pegRNAs and target sequences (FIG. 2).

In addition, in order to evaluate effects of factors other than the PBS length and RT template length on PE2 efficiency, the present inventors generated at least one library, termed Library 2, which includes 6,800 pairs of pegRNA-encoding sequences and their corresponding target sequences. Factors tested by using Library 2 include editing position, editing type (for example, insertion, deletion, or substitution), and positions of a two-position edit (FIG. 2).

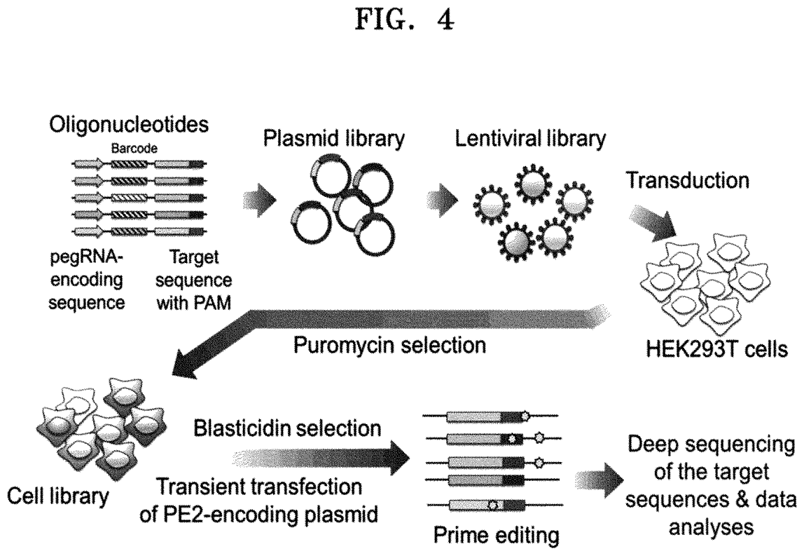

FIG. 4 is a schematic diagram of a high-throughput evaluation procedure of prime editing efficiency.

As shown in FIG. 4, HEK293T cells were transduced with the lentiviruses generated from the plasmid library to construct a cell library at MOI of 0.3, and untransduced cells were removed by puromycin selection. Each cell in this library expresses pegRNAs and includes a corresponding integrated target sequence. This cell library was then transfected with plasmids encoding PE2 and untransfected cells were removed by blasticidin selection. Four and a half days after the transduction with the PE2 plasmids, genomic DNA was isolated from the cells and PCR was performed to amplify the target sequences. The amplicons were deep sequenced to determine the frequency of mutations induced by PE2.

According to a Sanger sequencing analysis, 8.5% (=12/142) of the copies in the plasmid library contained one or more mutations in the guide sequence, scaffold, PBS, RT template, or target sequence region, which may be errors introduced during oligonucleotide synthesis and PCR amplification. In addition, when performing a high-throughput evaluation using lentiviral vectors, two distant factors may be mixed. As a result of measuring a non-binding rate between the pegRNA-encoding sequences and the barcode-target sequences in the cell library, the non-binding rate was found to be 4.2%. When it is expected that prime editing rarely occurs in these mutants or non-binding sequences, the observed PE2 efficiency would be 87% (=100%−8.5%−4.2%) of the actual PE2 efficiency. For example, when an actual PE2 efficiency is 25%, the observed PE2 efficiency would be 25%×87%=22%.

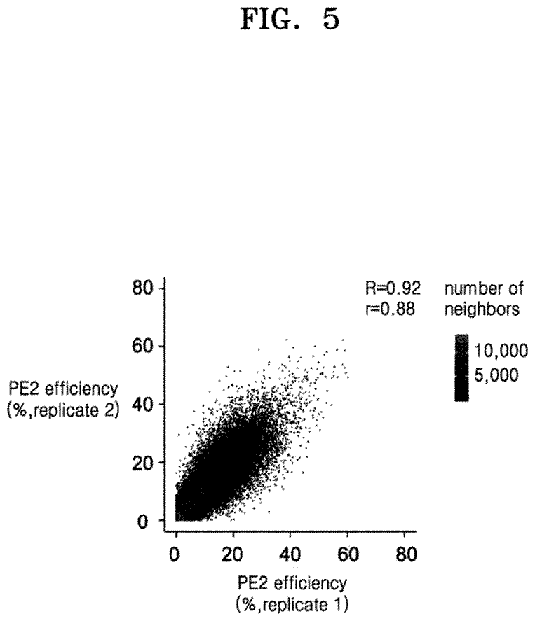

FIG. 5 shows a correlation of PE efficiencies of replicates independently transfected with PE2-encoding plasmids in two different experiments.

As shown in FIG. 5, a strong correlation between the replicates independently transfected by two different experiments was observed. Data from both replicates were combined for a subsequent analysis.

Next, a high-throughput approach was used to determine a correlation between the editing efficiency measured at integrated sequences and the editing efficiency at endogenous sites evaluated by individual tests.

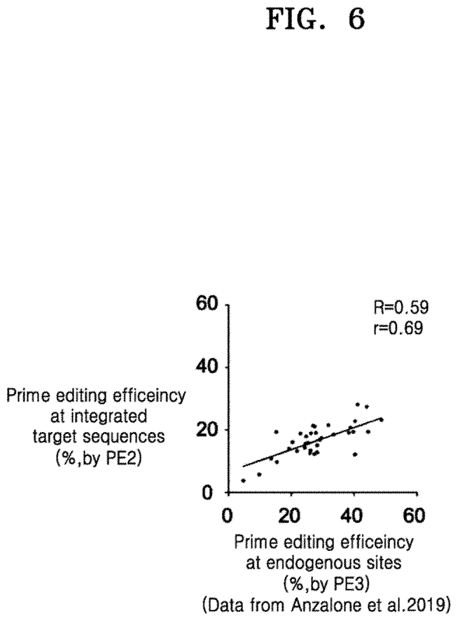

FIG. 6 shows a correlation between PE efficiencies measured at endogenous sites and PE efficiencies at the corresponding integrated target sequences.

As shown in FIG. 6, it was shown that Spearman's correlation coefficient (R)=0.59 and Pearson's correlation coefficient (r)=0.69 in the data set of the earlier study, indicating a strong correlation.

In addition, six new data sets of PE2 efficiency were generated at 20 to 31 endogenous sites randomly selected from the 54,836 pegRNAs of Libraries 1 and 2. The generated data sets were Endo-BR1-TR1, Endo-BR1-TR2, Endo-BR2-TR1, Endo-BR2-TR2, Endo-BR2-TR3, and Endo-BR3. In these experiments, plasmids encoding pegRNA and PE2 were transiently transfected.

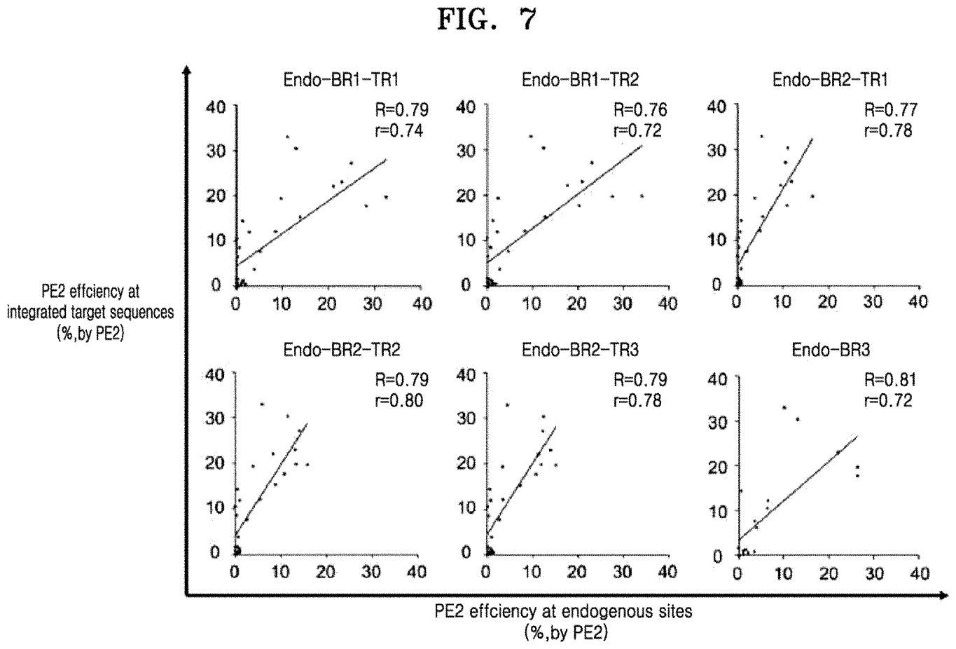

FIG. 7 shows a correlation between PE efficiency measured at endogenous sites and PE efficiency at the corresponding integrated target sequences.

As shown in FIG. 7, a high correlation was observed between the PE2 efficiency at the endogenous sites and the PE2 efficiency at the corresponding integrated target sequences.

### Experimental Example 2: Analysis of Prime Editing Efficiency Data

The collected prime editing efficiency data were analyzed.

For prime editing, Cas9 must make a nick by binding to a target sequence. Therefore, the activities of PE2-pegRNA and Cas9-sgRNA were expected to be highly correlated. The present inventors previously evaluated indel frequencies associated with Cas9-sgRNA activity in 2,000 target sequences (Kim, H. K. et al. SpCas9 activity prediction by DeepSpCas9, a deep learning-based model with high generalization performance. Sci Adv 5, eaax9249 (2019)).

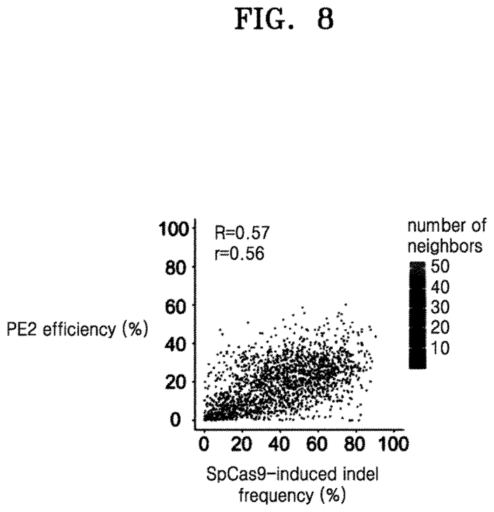

FIG. 8 shows a correlation between SpCas9-induced indel frequencies and PE2 efficiencies determined at the same target sequence.

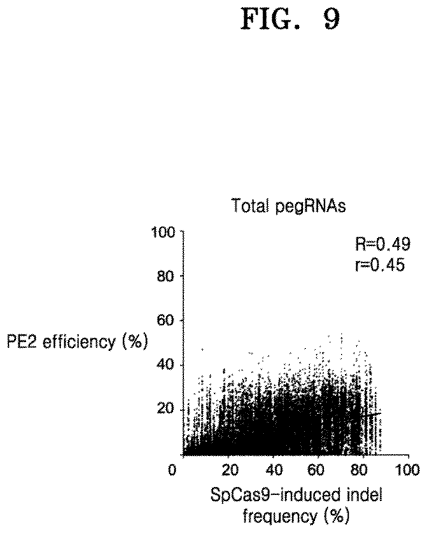

FIG. 9 shows a correlation between SpCas9-induced indel frequencies and PE2 efficiencies determined at the same target sequence by using Library 1.

As shown in FIGS. 8 and 9, when the correlation of the activities of PE2-pegRNA and Cas9-sgRNA on the same target sequence was evaluated, a moderate correlation was observed. The reason a moderate correlation was observed rather than a strong correlation was thought to be because prime editing requires additional processes unrelated to the indel-generating activity of Cas9. For example, these processes include reverse transcription of pegRNA, 5′ flap cleavage, and DNA repair.

For prime editing at a given target sequence, various combinations of PBS lengths and RT template lengths may be selected, and the lengths of these two regions in the pegRNA have a significant effect on the prime editing efficiency. Therefore, effects of different PBS lengths and RT template lengths on PE2 efficiency at 2,000 target sequences were evaluated next.

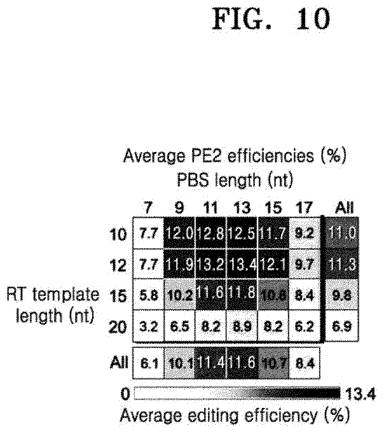

FIG. 10 shows effects of PBS lengths and RT template lengths on PE2 efficiency. The heatmap represents average editing efficiencies for given PBS lengths and RT template lengths.

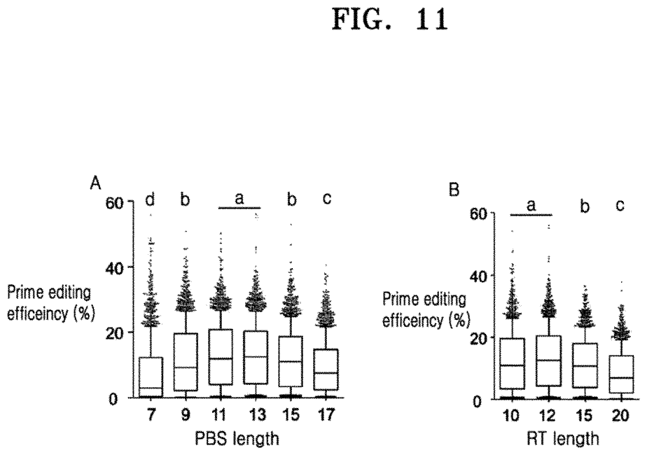

FIG. 11 shows effects of PBS lengths and RT template lengths on prime editing efficiency. (A) PE efficiency with PBSs of various lengths when a length of the RT template was fixed at 12 nt; (B) PE efficiency with RT templates of various lengths when a length of PBS was fixed at 13 nt.

As shown in FIGS. 10 and 11, a unimodal distribution was shown when an average editing efficiency was calculated for each combination of PBS lengths and RT template lengths; and the highest average efficiency (13.4%) was observed when pegRNAs having PBSs of 11 nts to 13 nts and RT templates of 10 nts to 12 nts were used.

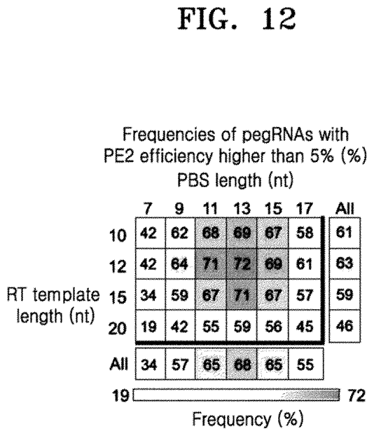

FIG. 12 shows frequencies of pegRNAs with PE2 efficiency of 5% or higher for a given PBS length and RT template length.

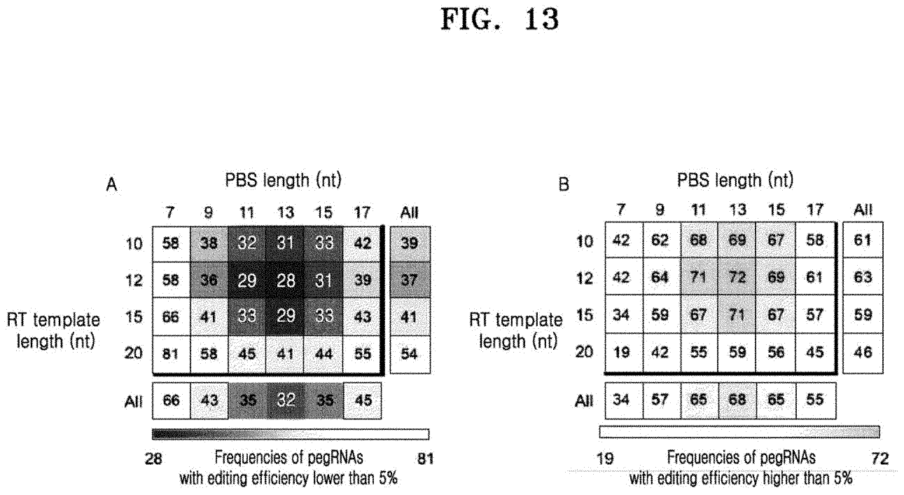

FIG. 13 shows (A) frequencies of pegRNAs with editing efficiency of less than 5% for a given PBS length and RT template length; and (B) frequencies of pegRNAs with editing efficiency of 5% or higher for a given PBS length and RT template length.

As shown in FIGS. 12 and 13, when defining poor pegRNA as having PE2 efficiency of less than 5% depending on the PBS length and RT template length, 28% to 81% (average of 43%) of pegRNAs fell into this category. In other words, 19% to 72% (average 57%) of pegRNA had PE2 efficiency of 5% or higher.

The present inventors have found that the optimal combination of a PBS length and an RT template length varies depending on a target sequence. Therefore, how often each combination of a PBS length and RT template length led to the highest editing efficiency for a given target sequence was evaluated next.

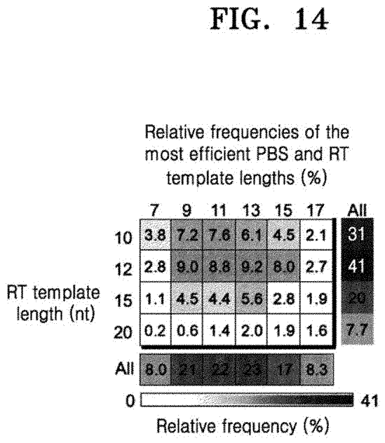

FIG. 14 shows frequencies of combinations of PBS lengths and RT template lengths that induce the highest editing efficiency for a given target sequence.

As shown in FIG. 14, these values also showed a unimodal distribution, and the highest editing efficiency was most frequently observed when PBSs of 9 nts to 13 nts and RT templates of 10 nts to 12 nts were used.

The present inventors also compared the average editing efficiency of each combination of PBS length and RT template length when selecting the most efficient pegRNA at each target.

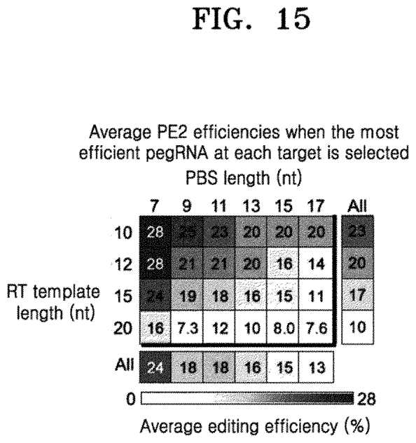

FIG. 15 shows average editing efficiencies when combinations of PBS lengths and RT template lengths that showed the highest editing efficiency for each target are selected.

As shown in FIG. 15, the average editing efficiencies at such optimal combinations of a PBS length and an RT template length were the highest when the lengths of the PBS and RT template were short (for example, 7-nt PBS and 10-nt to 12-nt RT template), and were decreased with increasing PBS lengths and RT template lengths.

Taken together, these results concluded that a use of 13-nt PBS and a 12-nt RT template for an initial test of PE2 efficiency, and extension to a use of 9-nt to 15-nt PBS and a 10-nt to 15-nt RT template for the second test are recommended.

### Experimental Example 3: Evaluation of Feature Importance

To evaluate other factors related to PE2 efficiency in a more systematic way, the Tree SHAP method (Lundberg, S. M. et al. From local explanations to global understanding with explainable Al for trees. Nature Machine Intelligence 2, 56-67 (2020)) was performed next by using 1,766 features including: melting temperature of various regions in pegRNA, GC count, GC content, minimum self-folding free energy, lengths of PBS and an RT template, DeepSpCas9 score (computationally predicted Cas9 nuclease activity at a given target sequence) (Kim, H. K. et al. SpCas9 activity prediction by DeepSpCas9, a deep learning-based model with high generalization performance. Sci Adv 5, eaax9249 (2019)), and direct sequence information such as all position-dependent and position-independent mono- and dinucleotides. When a high feature value was associated with high prime editing efficiency, the feature was classified as a favored feature; and when a high feature value was associated with low prime editing efficiency, the feature was classified as an unfavored feature.

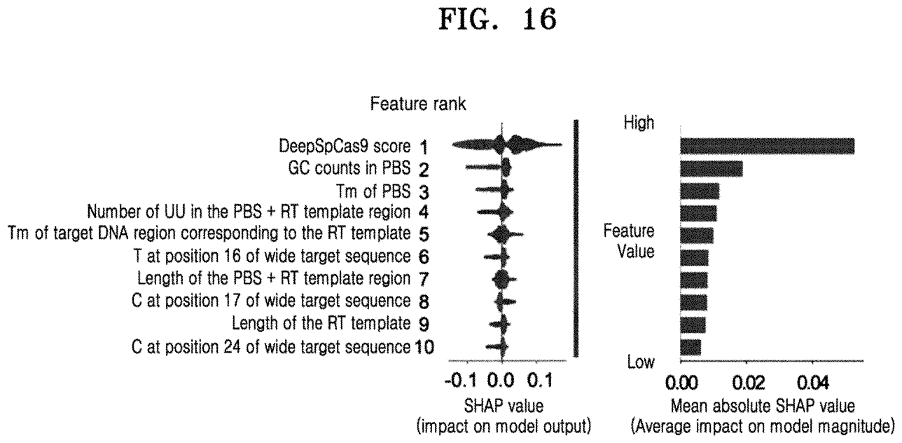

FIG. 16 shows the 10 most important features associated with PE2 efficiency, determined by Tree SHAP (XGBoost classifier).

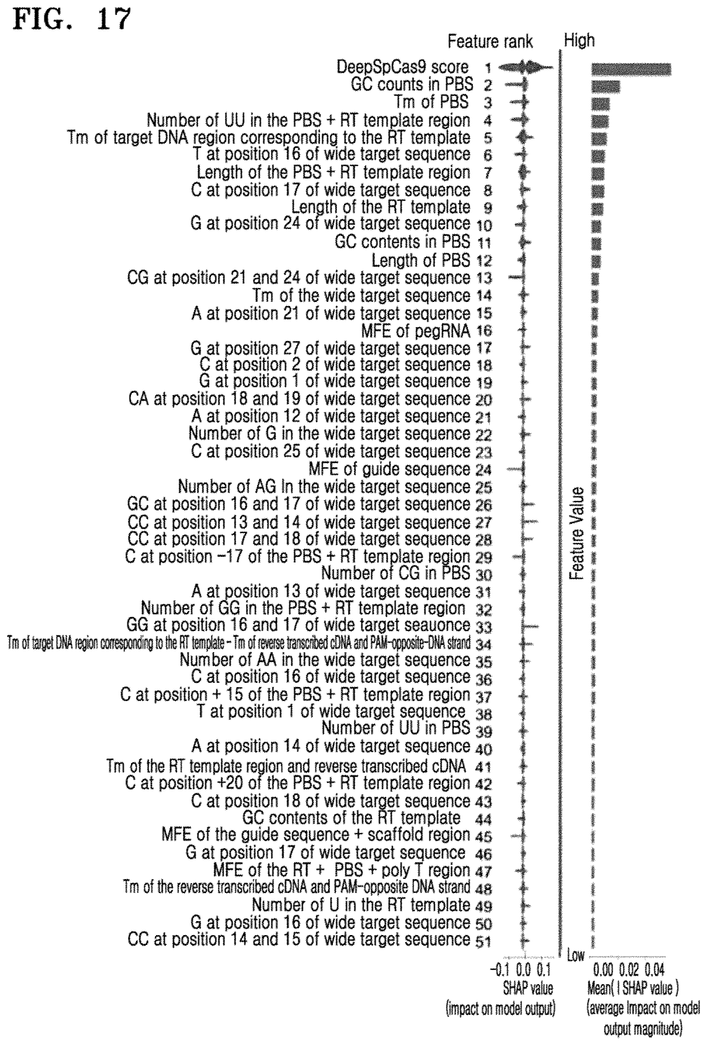

FIG. 17 shows the 1st to 51st most important features associated with PE2 efficiency, as determined by the Tree SHAP.

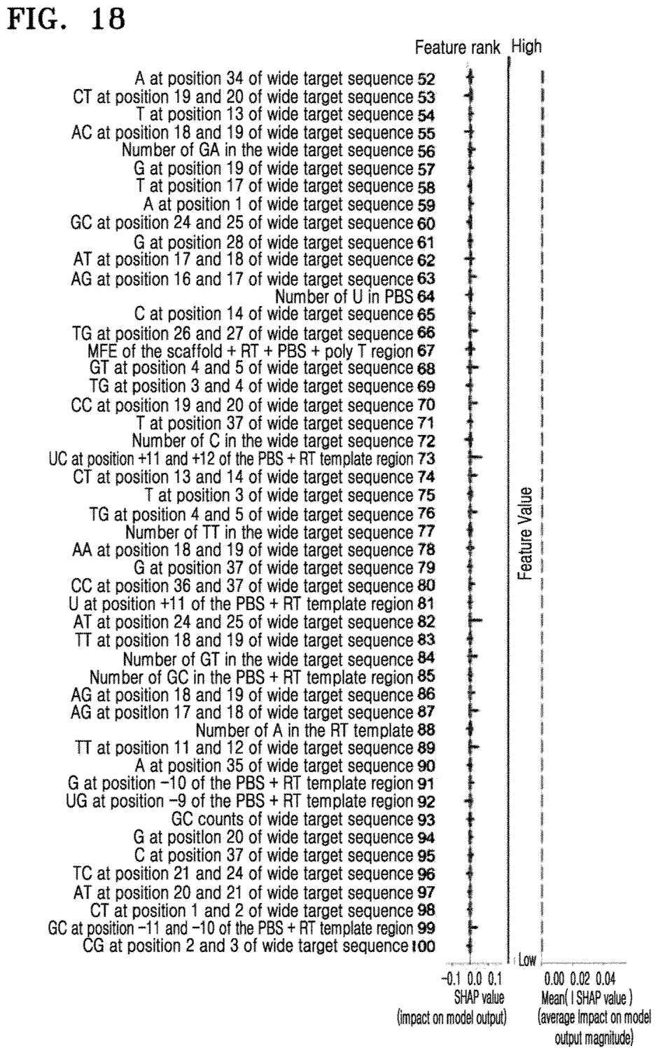

FIG. 18 shows the 52nd to 100th most important features associated with PE2 efficiency, as determined by the Tree SHAP.

The first important feature was a DeepSpCas9 score at the corresponding target sequence (favored) (FIG. 16), which is consistent with the correlation between SpCas9-induced indel frequency and PE2 efficiency, as shown above.

GC count in PBS (favored) was the second most important feature. Along with this result, GC content in PBS (favored) was the 11th most important feature (FIG. 17). The GC content may be calculated by dividing the GC count (number of G or C nucleotides) by the length of the related DNA strand. According to this result, it may be seen that a high GC content in PBS results in strong binding of pegRNA to the nick strand of the target DNA, which is required for reverse transcription.

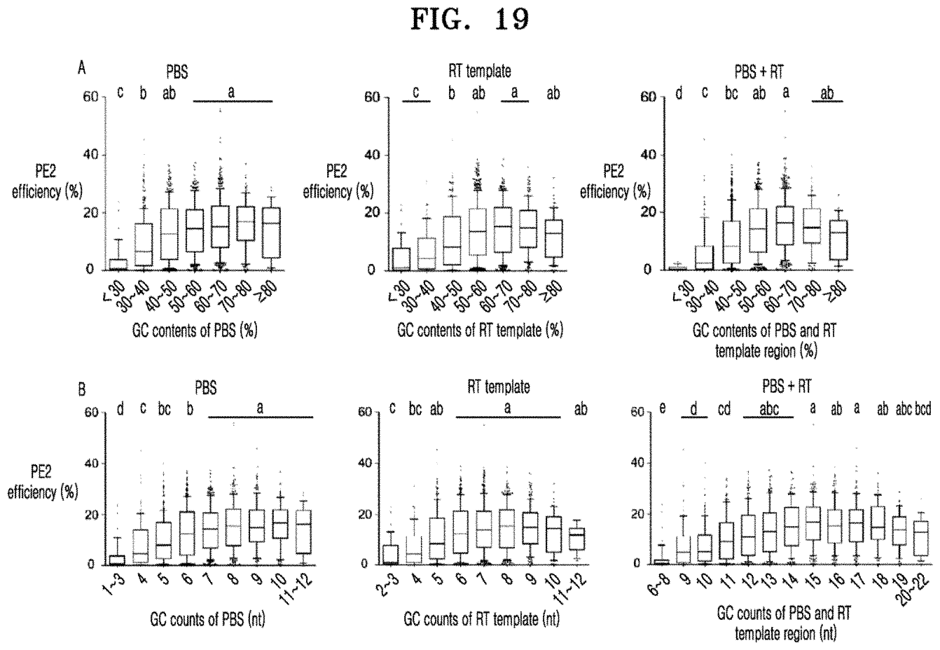

FIG. 19 shows effects of GC content and GC count in PBS and an RT template on prime editing efficiency.

As shown in FIG. 19, when the effects of GC content and GC count in PBS, an RT template, and a combination of PBS and an RT template on PE2 efficiency were systematically evaluated, it was clearly observed that as the GC content and GC count of PBS increased, the PE2 efficiency became higher. When the GC content of PBS was less than 30%, relatively high editing efficiency was shown at lengths as long as 15 nts, but PE2 efficiency was poor for all tested PBS lengths. Conversely, shortening the PBS lengths to 7 nts to 11 nts resulted in relatively high PE2 efficiency, when the GC content of PBS was 60% or higher. Based on these results, it is recommended to use PBS with a length of 15 nts or 9 nts, respectively, when the GC content is less than 40% or greater than 60%, respectively.

However, the GC content and GC count of the RT template had only a slight effect on the PE2 efficiency, and the PE2 efficiency tended to decrease when the GC-related parameters were extremely high or low. Consistent with these results, GC content or GC count of an RT template was not included in the 40 most important features.

The third and fifth most important features were a melting temperature of PBS (favored) and a melting temperature of a target DNA region corresponding to the RT template (that is, the opposite strand to the strand containing the protospacer adjacent motif (PAM), referred to herein as “PAM-opposite strand”; this feature is disfavored only when the melting temperature is higher than 35° C.). A high melting temperature of PBS is likely associated with a high GC count in PBS, coupled with strong binding of the PBS region of pegRNA to the target DNA, which will promote a reverse transcription reaction.

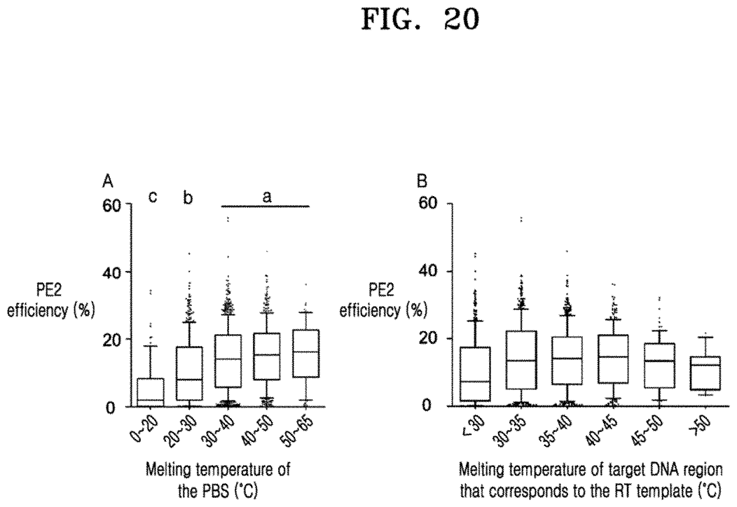

FIG. 20 shows effects of a melting temperature of PBS, and a target DNA region that corresponds to an RT template on prime editing efficiency.

As shown in FIG. 20, as a result of examining the relationship between PE2 efficiency and a PBS melting temperature, it was confirmed that the PE2 efficiency also increased as the melting temperature of PBS increased. When the melting temperature of the target DNA region corresponding to the RT template is too high, conversion of the 3′ flap to the 5′ flap, that is, the process required to integrate the reverse transcribed DNA sequence into the genome, may be prevented. The relationship between PE2 efficiency and the melting temperature in this region was analyzed, and it was confirmed that when the melting temperature increased to 35° C. or higher, the PE2 efficiency tended to decrease, although the difference was not statistically significant.

The fourth important feature was the number of UUs in the RT+PBS domain (disfavored). This feature is due to multiple Ts in the pegRNA-encoding sequence corresponding to the multiple Us in the pegRNA that may reduce efficiency of transcription by an RNA polymerase III, thereby reducing an intracellular pegRNA concentration.

The sixth and eighth most important features were respectively the presence of T at position 16 (disfavored) and the presence of C at position 17 (favored) in the wide target sequence (position 1 is the 20th nucleotide from NGG PAM). According to a previous study, T at position 16 is associated with a reduced Cas9 nuclease activity. In addition, T at position 16 reduces the GC count in PBS, which is undesirable for reverse transcription, especially when a length of PBS is short. Combining these two effects makes T at position 16 the sixth most important feature. Similarly, according to a previous study, a Cas9 nuclease activity increased when A or C was at position 17. In addition, C at position 17 increases the GC count in PBS, facilitating reverse transcription. The combination of these two effects makes C at position 17 a favored feature.

The seventh, ninth, and twelfth most important features were a RT length and PBS length (generally disfavored), RT template length (disfavored only when long), and PBS length (generally disfavored).

The tenth most important feature is G at position 24 in the wide target sequence (disfavored). The intended edit (+5, G to C) would replace G at position 22, which would result in a PAM edit, preventing Cas9 from rebinding to the target sequence.

### Experimental Example 4: Evaluation of Prime Editing Efficiencies for Various Types of Editing

Next, using 6,800 pairs of pegRNAs and target sequences (=200 target sequences ×1 PBS/target sequence ×34 RT templates/target sequences) from Library 2, PE2 efficiency was evaluated for more diverse types of genome editing, and effects of types of genome editing (that is, generation of indels vs. substitutions), edited positions, and numbers of inserted or deleted nucleotides on PE2 efficiency were determined.

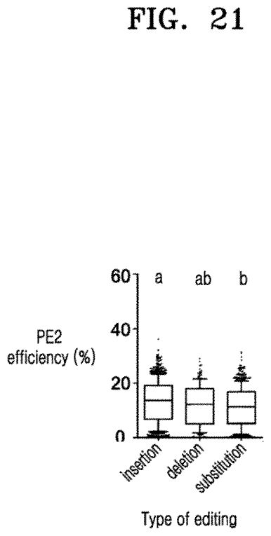

FIG. 21 shows PE2 efficiencies for insertions, deletions, and substitutions of 1-bp.

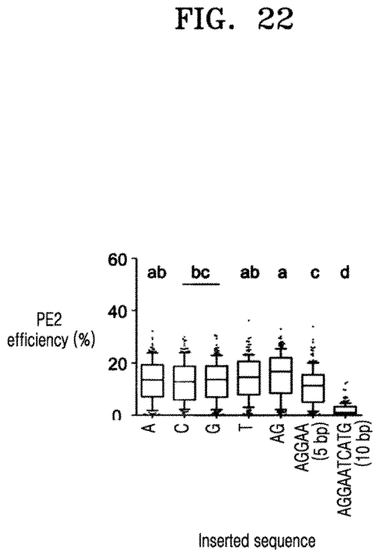

FIG. 22 shows effects of types and numbers of inserted nucleotides on PE2 efficiency.

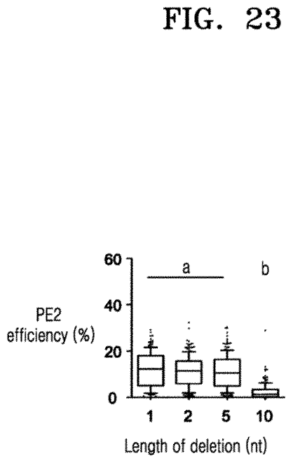

FIG. 23 shows effects of deletion lengths on PE2 efficiency.

First, effects of generating 1-bp insertions, 1-bp deletions, and 1-bp substitutions were evaluated. In general, the efficiencies may be ranked as insertion≥deletion≥substitution, and it was confirmed that the difference of efficiencies between insertion and substitution was statistically significant (FIG. 21).

Then, effects of the types and numbers of inserted nucleotides on prime editing-induced insertion were evaluated. It was confirmed that the identity of the inserted nucleotide did not affect the 1-bp insertion efficiency. When the number of inserted nucleotides was increased from 1 bp to 2 bp, 5 bp, and 10 bp, the insertion efficiency was similar for 1-bp and 2-bp insertions, decreased for 5-bp insertions, and significantly decreased for 10-bp insertions (FIG. 22).

At the same time, PE efficiencies for 1-, 2-, 5-, and 10-bp deletions were evaluated, and PE efficiencies were similar for 1-, 2-, and 5-bp deletions, and significantly different for 10-bp deletions (FIG. 23).

Next, an effect of identity of the substituted nucleotides on PE2 efficiency was investigated.

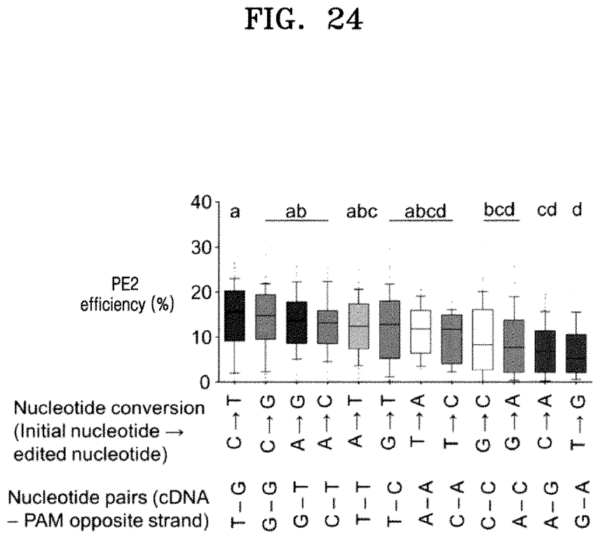

FIG. 24 shows effects of substitution types on PE2 efficiency.

As shown in FIG. 24, all 12 possible types of 1-bp substitutions at position +1 from the nicking site, corresponding to positions 17 and 18 in the wide target sequence, were tested, and the PE2 efficiency was found to be slightly different depending on the type of substitution; C to T conversion and T to G conversion showed the highest PE2 efficiency and lowest PE2 efficiency, respectively. To gain mechanistic insight into these effects, temporary base pairing between nucleotides in cDNA generated from the RT template and the corresponding nucleotides on the PAM-opposite strand was considered. Interestingly, the PE2 efficiencies were ranked as follows: T (cDNA)-G (corresponding nucleotide on the PAM-opposite strand) and G-T pair C-T and T-C pairs ≥C-A and A-C pairs ≥A-G and G-A pairs. Here, the differences between the T-G and G-T pair groups and the A-G and G-A pair groups were statistically significant, suggesting that temporary base pairing between cDNA and a PAM-opposite strand may affect PE2 efficiency. When temporary base pairs are formed between identical nucleotides, for example, T (cDNA)-T (corresponding nucleotide in the PAM-opposite strand), G-G, C-C, and A-A, which respectively correspond to A to T, C to G, G to C, and T to A conversions, PE2 efficiencies were all similar.

In addition, PE2 efficiencies were analyzed for these four conversions mediated by temporary base pairs between the same nucleotides at different positions, such as +9, +11, and +14 from the nicking site.

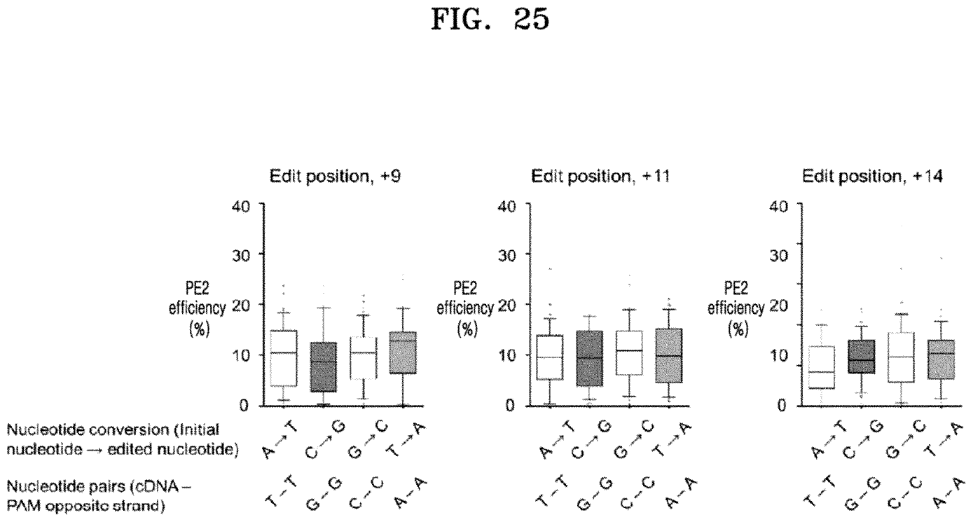

FIG. 25 shows effects of substitution types on prime editing efficiency.

As shown in FIG. 25, all 3 tested positions were similar for 4 tested conversions, which were similar to the analysis at position +1 from the nicking site.

In addition, effects of editing positions on the 1-bp substitution efficiency were investigated.

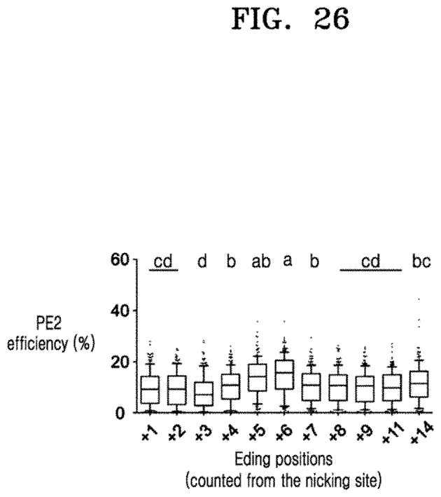

FIG. 26 shows effects of editing positions on PE2 efficiency for substitutions of 1-bp conversions.

As shown in FIG. 26, editing efficiencies were generally similar at all tested positions ranging from +1 to +14 from the nicking site except for positions +3, +5, and +6. The lowest editing efficiency was observed at position +3, although the underlying mechanism for this effect is not clear. The highest editing efficiency was observed at positions +5 and +6, and the position of GG PAM; as described above, when PAM is not edited, Cas9 may recombine to the target sequence and nick the reverse transcribed DNA strand before repair of the complementary strand, reducing PE efficiency.

This effect of PAM editing on PE efficiency may also be observed when 2-bp substitution efficiency is evaluated.

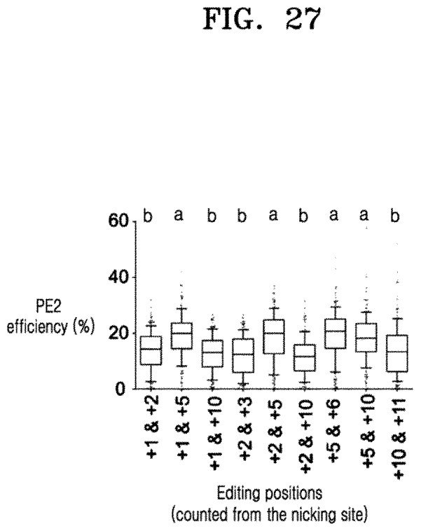

FIG. 27 shows effects of editing positions on prime editing efficiency for substitutions of 1-bp conversions at two positions.

As shown in FIG. 27, 2-bp substitutions were generated at various positions, and the editing efficiency was higher when one or two nucleotides (positions 5 and 6) were edited in PAM (for example, positions 1 and 5, positions 2 and 5, positions 5 and 6, and positions 5 and 10), than when PAM was left intact (positions 1 and 2, positions 1 and 10, positions 2 and 3, positions 2 and 10, or positions 10 and 11 edited).

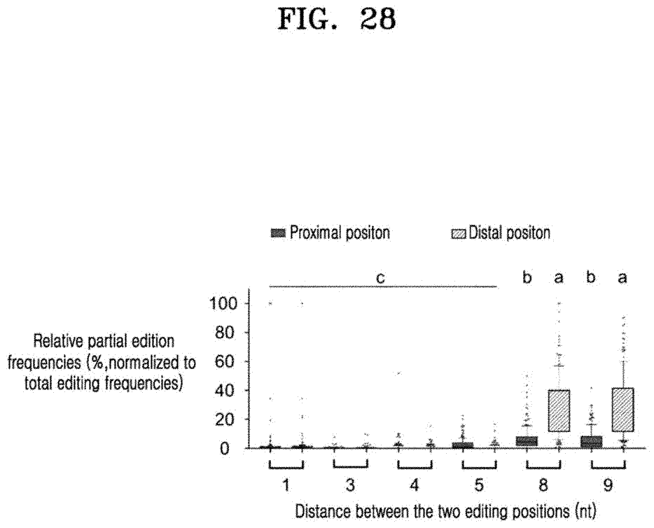

FIG. 28 shows relative partial editing frequencies according to a distance between the two editing positions described in FIG. 27.

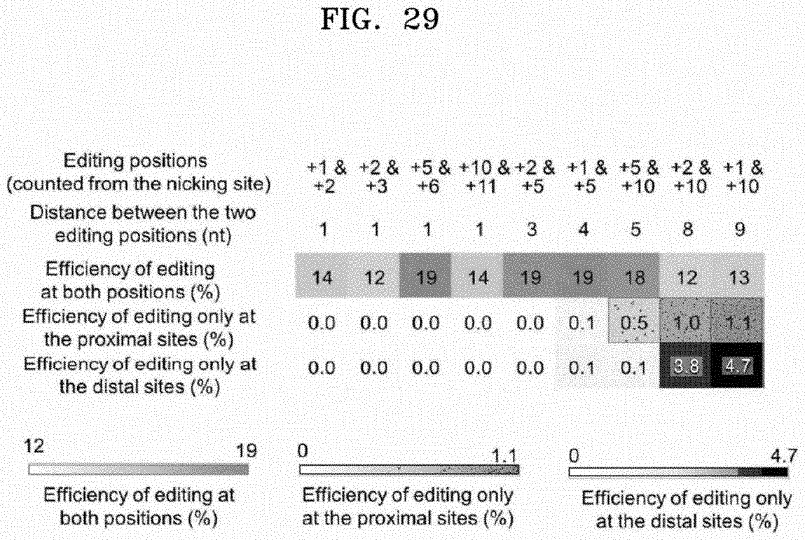

FIG. 29 shows results of an analysis of prime editing when two nucleotides were to be substituted.

When an editing position affects PE2 efficiency, using an SpCas9 variant that recognizes different PAM instead of wild-type SpCas9 may improve the PE2 efficiency at the same target sequence. Interestingly, a maximum of 20% of the sequences, in which at least one of the two intended edits was introduced, had only one edit (FIGS. 28 and 29). The partial editing rate was higher at positions far from the nicking site than at positions close to the nicking site, and showed a tendency to increase as the distance between the two positions increased.

### Experimental Example 5: Verification of Deep Learning-Based Predictive Models 1

(1) Creation of a Model DeepPE for Predicting PE2 Efficiency According to PBS Lengths and RT Template Lengths in Certain Types of Editing

According to Example 2-6, a computational model to predict PE2 efficiency at a given target sequence paired with 24 different pegRNAs having variable PBS lengths and RT template lengths was developed.

The PE efficiencies obtained by using Library 1 with 48,000 pairs of pegRNAs and target sequences were divided into two data sets by random sampling and named HT-Training (n=38,692) and HT-Test (n=4,457), respectively. In this regard, the same target sequence was not shared between the two data sets. A computational model was created for predicting PE2 efficiency at a given target sequence paired with 24 pegRNAs having different combinations of PBS lengths and RT template lengths by using HT-training as training data, when the prime editing is designed for G to C conversion at position +5.

(2) Performance Verification

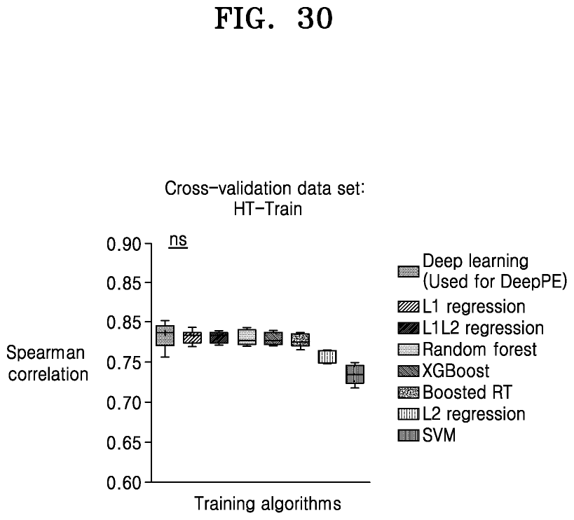

FIG. 30 shows cross-validation results of predictive models according to machine learning frameworks used.

As shown in FIG. 30, the cross-validation results showed that the deep learning framework had the highest performance, although the difference from boosted RT, which was the second best framework, was not statistically significant.

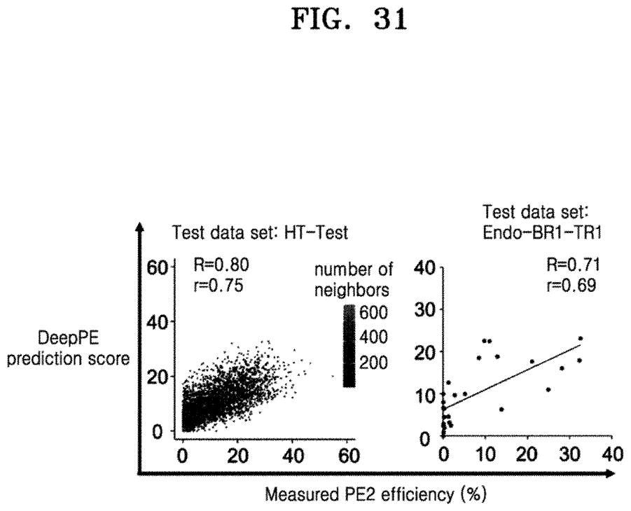

FIG. 31 shows evaluation results of DeepPE using the data sets HT-Test (number of pairs of pegRNAs and target sequences n=4,457) and Endo-BR1-TR1 (n=26).

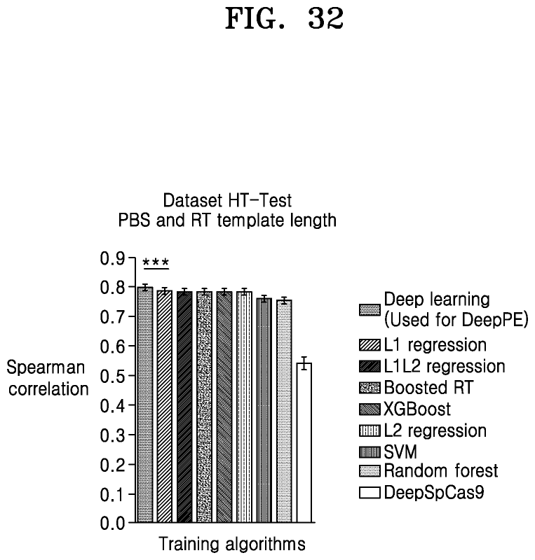

FIG. 32 is results of comparing the performance of DeepPE with that of other prediction models using the dataset HT-Test.

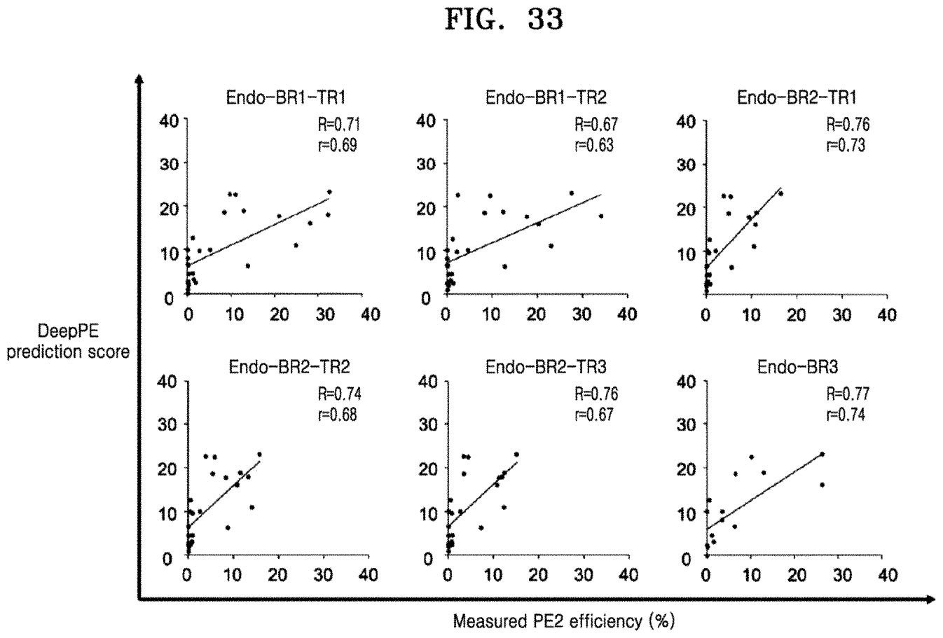

FIG. 33 shows evaluation results of DeepPE using six data sets obtained by measuring PE2 efficiency at endogenous site after transient transfection of HEK293T cells with a plasmid encoding pegRNA and PE2.

As shown in FIGS. 31 to 33, as a result of evaluating by using HT-test as a test data set, DeepPE, a deep learning-based model, outperformed other models based on existing machine learning. As a result of testing by using six replicates of PE2 efficiency at endogenous sites as test data sets, the Spearman and Pearson correlation coefficients (R and r) were R=0.67 to 0.77 (mean 0.73) and r=0.63 to 0.74 (mean 0.69), respectively, indicating a good performance of DeepPE in predicting PE2 efficiency at endogenous sites.

DeepPE was evaluated in two additional cell types, HCT116 and MDA-MB-231, at target sequences that were never used for training DeepPE.

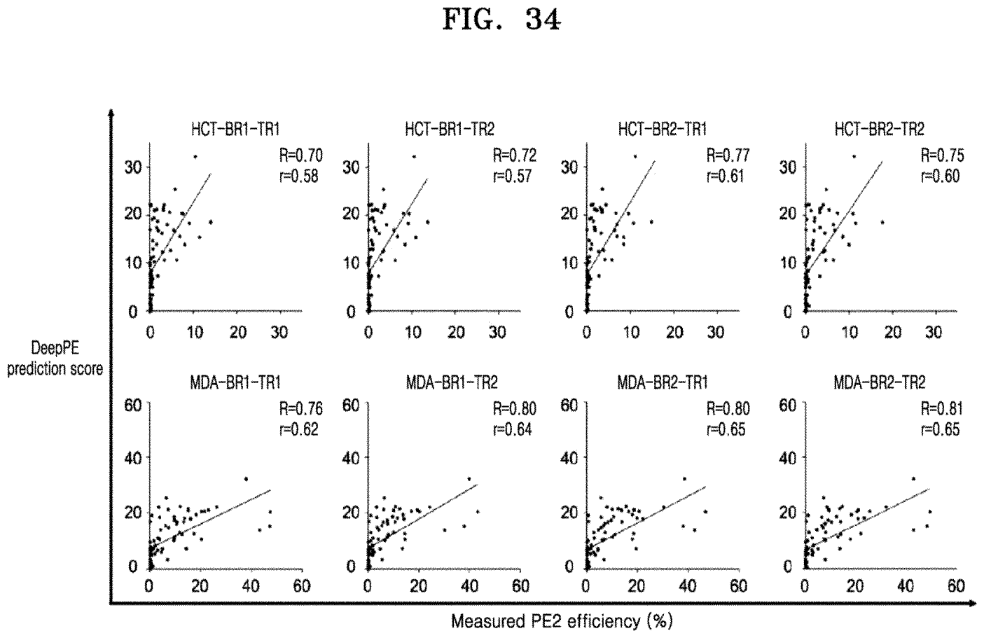

FIG. 34 shows evaluation results of DeepPE using HCT116 and MDA-MB-231 cells.

As shown in FIG. 34, DeepPE showed excellent performance in all biological and technical replicates. It was found that for HCT116 cells, R=0.70 to 0.77 (mean 0.74), and r=0.57 to 0.61 (mean 0.59); and for MDA-MB-231 cells, R=0.76 to 0.81 (average 0.79), and r=0.62 to 0.65 (average 0.64).

The usefulness of DeepPE for selecting the most efficient combination (out of 24 possible combinations) of PBS lengths and RT template lengths for a given target sequence was confirmed.

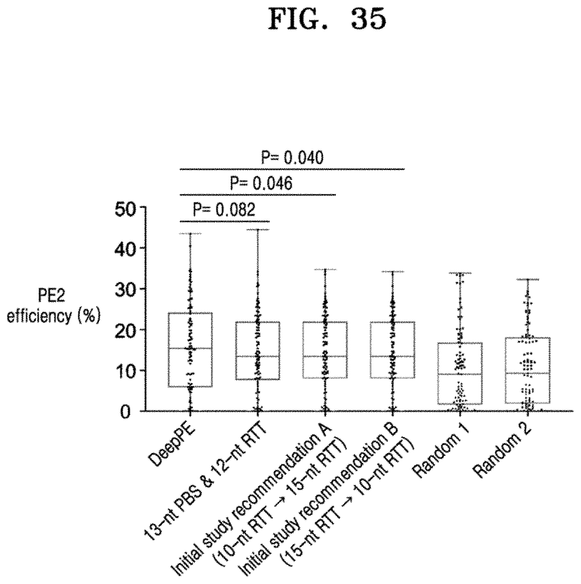

FIG. 35 shows a comparison of the performance of DeepPE with that of other methods for selecting the most efficient combination out of 24 possible combinations of PBS lengths and RT template lengths at a given target sequence. For example, “13-nt PBS & 12 nt-PT template” means selecting a combination of these lengths regardless of the target sequence. Recommendations A and B of an earlier study are based on using 13-nt PBS and 12-nt RT template (RTT) and not using G as the last template nucleotide by changing the RTT length as needed. In recommendation A, when the last template nucleotide is G, 10-nt RTT is chosen over 12-nt RTT. After such a change, when the last template nucleotide is G again, 15-nt RTT is selected. In recommendation B, when the last template nucleotide is G, 15-nt RTT is chosen over 12-nt RTT. After such a change, when the last template nucleotide is G again, 10-nt RTT is selected. As control groups, pegRNAs were randomly selected (Random 1 and Random 2).

As shown in FIG. 35, the average absolute and relative PE2 efficiencies when using DeepPE were 1.2% and 8.3%, respectively. This was significantly higher than the efficiency obtained by using the recommendations based on an earlier study (that is, using 13-nt PBS and 12-nt RT templates, and not using G as the last template nucleotide).

Also, for intended editing, there may be multiple target sequences; in this case, DeepPE will be useful for selecting a target sequence that may be edited with the highest efficiency.

### Experimental Example 6: Verification of Deep Learning-Based Predictive Models 2

(1) Creation of Models PE_Type and PE_Position for Predicting PE2 Efficiency According to Editing Types and Positions

According to Example 2-6, a computational model PE_Type for predicting PE2 efficiency according to editing types and a computational model PE_position for predicting PE2 efficiency according to editing positions were developed using the data set obtained by using Library 2.

The data obtained by using Library 2 were divided into Type-training, Type-test, Position-training, and Position-test to ensure that target sequences were not shared between the training and test data sets.

(2) Performance Verification

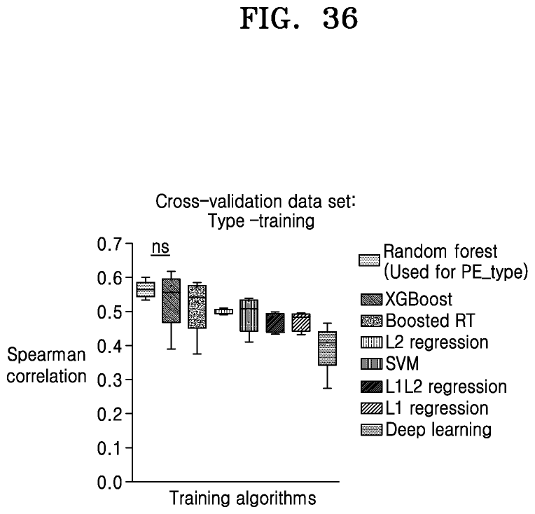

FIG. 36 shows cross-validation results of PE-type according to machine learning frameworks used.

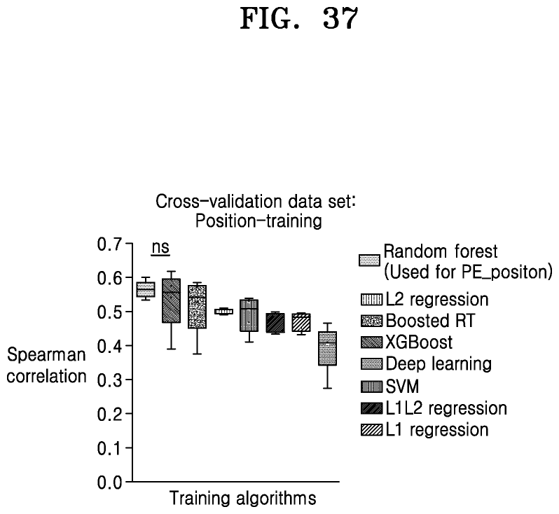

FIG. 37 shows cross-validation results of PE_position according to machine learning frameworks used.

As shown in FIGS. 36 and 37, as a result of cross-validation using Type-training and Position-training, random forest had the best performance, but the difference with the second best framework was not statistically significant. In both cases, deep learning showed limited performance due to the relatively small number of target sequences and pegRNAs. When evaluated by using Type-test and Position-test, PE_type and PE_position, the random forest-based model showed useful performance. PE_type, R=0.47, r=0.48; PE_position, R=0.56, r=0.56.

Therefore, evaluating prime editing efficiency at a larger number of target sequences by using pegRNAs with all possible PBS lengths and RT template lengths and a greater variety of intended edits would yield more useful models.

The Present inventors provide a web tool at http://deepcrispr/DeepPE that provides the results of DeepPE, PE_type, and PE_position for a given target sequence. When a sequence that includes a target sequence is input, the web tool identifies candidate target sequences and provides expected PE2 efficiencies for a total of 57 pegRNAs per target sequence (24 pegRNAs in DeepPE, 23 pegRNAs in PE_type, and 10 pegRNAs in PE_position).

Prime editing is revolutionary in that it allows small genetic mutations to be introduced in a highly efficient manner without using donor DNA. Information on factors influencing PE2 efficiency identified in this study based on a high-throughput analysis along with DeepPE, PE_type, and PE_position is expected to facilitate prime editing.

As above, the present inventors performed high-throughput evaluation of prime editor 2 (PE2) activity in human cells by using 54,836 pairs of pegRNAs and target sequences. By using a large data set of PE2 efficiencies, i) computational models predicting PE2 efficiency for a total of 57 pegRNAs, which have PBSs and RT templates of different lengths, and are designated to generate different types of intended edits at different positions at a given target sequence, were developed and ii) multiple factors affecting PE2 efficiency were identified in a highly systematic manner. The computational model and information on PE2 efficiency will facilitate prime editing.

1. A system for predicting prime editing efficiency by using deep learning, comprising:
an information input unit that receives an input of data on prime editing efficiency of a prime editor;
a predictive model generator for generating prime editing efficiency predictive models by performing deep learning to learn a relationship between features affecting prime editing efficiency and prime editing efficiency, by using the data received from the information input unit;
a candidate sequence input unit that receives an input of a candidate target sequence for prime editing; and
an efficiency predictor for predicting prime editing efficiency by applying the candidate target sequence input into the candidate sequence input unit to an efficiency predictive model generated in the predictive model generator.

2. The system for predicting prime editing efficiency by using deep learning of claim 1, wherein the prime editor is prime editor 2.

3. The system for predicting prime editing efficiency by using deep learning of claim 1, wherein the prime editing efficiency is represented by a rate of occurrence of intended edits by a prime editor and pegRNA at a target sequence without generation of an unintended mutant.

4. The system for predicting prime editing efficiency by using deep learning of claim 1, wherein the data on prime editing efficiency is obtained by performing a method comprising:
introducing a prime editor into a cell library including an oligonucloeitde including a nucleotide sequence encoding pegRNA and a target nucleotide sequence targeted by the pegRNA;
performing deep sequencing by using DNA obtained from the cell library into which the prime editor has been introduced; and
analyzing prime editing efficiency from data obtained by the deep sequencing.

5. The system for predicting prime editing efficiency by using deep learning of claim 4, wherein the oligonucleotide further comprises a barcode sequence.

6. The system for predicting prime editing efficiency by using deep learning of claim 1, wherein the features affecting prime editing efficiency are extracted from information about pegRNA and a target sequence.

7. The system for predicting prime editing efficiency by using deep learning of claim 6, wherein the information about the pegRNA and the target sequence comprises at least one of information about a reverse transcriptase (RT) template sequence, information about a primer binding site (PBS) sequence, and information about the target sequence.

8. The system for predicting prime editing efficiency by using deep learning of claim 1, wherein the predictive model generator comprises a feature extraction module for extracting features affecting prime editing efficiency from information about pegRNA and a target sequence.

9. The system for predicting prime editing efficiency by using deep learning of claim 1, wherein the predictive model generator performs deep learning based on a convolutional neural network (CNN) or a multilayer perceptron (MLP).

10. The system for predicting prime editing efficiency by using deep learning of claim 1, wherein the candidate target sequence comprises a protospacer adjacent motif (PAM), and a protospacer sequence.

11. The system for predicting prime editing efficiency by using deep learning of claim 1, wherein the efficiency predictor predicts prime editing efficiency of candidate target sequences by a prime editor and pegRNA.

12. The system for predicting prime editing efficiency by using deep learning of claim 1, further comprising an output unit for outputting the prime editing efficiency predicted by the efficiency predictor.

13. A method of building a system for predicting prime editing efficiency by using deep learning, comprising:
obtaining a prime editing efficiency data set of a prime editor; and
generating prime editing efficiency predictive models by performing deep learning to learn a relationship between features affecting prime editing efficiency and prime editing efficiency, by using the prime editing efficiency data set.

14. The method of building a system for predicting prime editing efficiency by using deep learning of claim 13, wherein the obtaining the efficiency data set comprises:
introducing a prime editor into a cell library including an oligonucloeitde including a nucleotide sequence encoding pegRNA and a target nucleotide sequence targeted by the pegRNA;
performing deep sequencing by using DNA obtained from the cell library into which the prime editor has been introduced; and
analyzing prime editing efficiency from data obtained by the deep sequencing.

15. The method of building a system for predicting prime editing efficiency by using deep learning of claim 13, wherein the prime editing efficiency is calculated by a rate of occurrence of intended edits by a prime editor and pegRNA at a target sequence without generation of an unintended mutant.

16. The method of building a system for predicting prime editing efficiency by using deep learning of claim 13, wherein the features affecting prime editing efficiency are extracted from information about pegRNA and a target sequence.

17. The method of building a system for predicting prime editing efficiency by using deep learning of claim 16, wherein the information about the pegRNA and the target sequence comprises at least one of information about an RT template sequence, information about a PBS sequence, and information about the target sequence.

18. The method of building a system for predicting prime editing efficiency by using deep learning of claim 13, wherein in the generating of the predictive models, deep learning is performed based on a convolutional neural network (CNN) or a multilayer perceptron (MLP).

19. A method of predicting prime editing efficiency, comprising:
designing candidate target sequences for prime editing; and
predicting prime editing efficiency by applying the designed candidate target sequences to the system for predicting prime editing efficiency of claim 1.

20. A computer-readable recording medium on which a program for executing the method according to claim 19 on a computer is recorded.
